(19) United States
(12) Patent Application Publication
(10) Pub. No.: US 2022/0267321 A1
(43) Pub. Date: Aug. 25, 2022

(54) AZAINDOLE PYRAZOLE COMPOUNDS AS CDK9 INHIBITORS

(71) Applicant: MEDSHINE DISCOVERY INC., Nanjing (CN)

(72) Inventors: Yingchun LIU, Shanghai (CN); Zhaobing XU, Shanghai (CN); Lihong HU, Shanghai (CN); Charles Z. Z. DING, Shanghai (CN); Wen JIANG, Shanghai (CN); Jian LI, Shanghai (CN); Shuhui CHEN, Shanghai (CN)

(21) Appl. No.: 17/622,570

(22) PCT Filed: Jun. 24, 2020
(86) PCT No.: PCT/CN2020/098002

§ 371 Date: Dec. 23, 2021

(30) Foreign Application Priority Data
Jun. 27, 2019 (CN) 201910566823.3

(51) Int. Cl. C07D 471/04; A61P 35/00

(52) U.S. Cl. CPC C07D 471/04; A61P 35/00

(57) ABSTRACT

The present invention discloses new azaindole pyrazole compounds as CDK9 inhibitors, in particular, compounds as represented by formula (I), pharmaceutically acceptable salts and isomers thereof, and the use of compounds represented by formula (I), pharmaceutically acceptable salts and isomers thereof, and the pharmaceutical composition containing them in the preparation of cancer drugs.

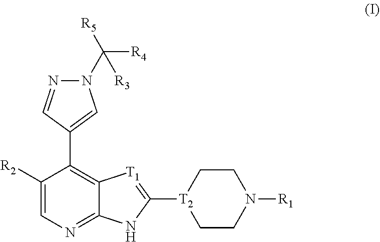

### CROSS-REFERENCE TO RELATED APPLICATION

This application claims the following priority:

Application No.: CN201910566823.3, application date: Jun. 27, 2019.

### TECHNICAL FIELD

The present invention relates to new azaindole pyrazole compounds as CDK9 inhibitors, and specifically to compounds represented by formula (I), pharmaceutically acceptable salts and isomers thereof, and the use of compounds represented by formula (I), pharmaceutically acceptable salts and isomers thereof, and the pharmaceutical composition containing them in the preparation of cancer drugs.

### BACKGROUND

The occurrence of tumors is often accompanied by excessive activation and continuous proliferation of cells, and CDK (Cell Cycle Dependent Kinase) plays an important role in regulating the cell cycle and transcription process under the regulation of intracellular and extracellular signals. In cancer cells, the activity of CDK-cyclin (cyclin) is often dysregulated. Possible reasons include: overactivation of signal transduction pathways, overexpression of cyclin, abnormal expansion of CDK, and inactivation or absence of endogenous inhibitors, which inspire people to develop tumor treatment technologies by constantly looking for new CDK inhibitors.

CDK9 is a member of the CDK family, mainly involved in the process of transcription regulation. The heterodimer composed of CDK9 and cyclin (T1, T2a, T2b, K) participates in the formation of positive transcription elongation factor (p-TEFb), of which there are approximately 80% of CDK9 binds to cyclinT1. P-TEFb regulates transcription elongation by phosphorylating the carboxyl terminal domain of RNA polymerase II, mainly ser-2 phosphorylation. Inhibition and transcriptional repression of CDK9 lead to rapid consumption of short-lived mRNA transcripts and related proteins (including Myc and Mcl-1), resulting in the death of cancer cells highly dependent on these anti apoptotic proteins. Therefore, targeting CDK9 represents a treatment strategy for tumor types that are highly dependent on these anti-apoptotic proteins.

At present, CDK9 small molecule inhibitors have entered the clinical research stage for cancer treatment, namely Bayer's BAY1251152 and AstraZeneca's AZD4573. Related patents include WO2012160034, WO2014076091, WO2009047359, WO2011110612, US2016376287.

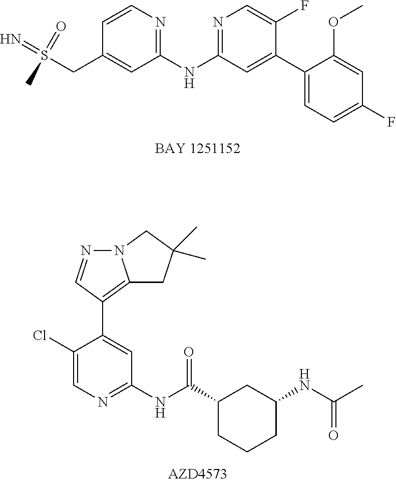 [SMILES: COc1cc(F)ccc1-c1cc(Nc2cc(C[S@](C)(=N)=O)ccn2)ncc1F ; CC(=O)N[C@@H]1CCC[C@H](C(=O)Nc2cc(-c3cnn4c3CC(C)(C)C4)c(Cl)cn2)C1]

Although many efforts have been made to develop CDK9 inhibitors for the treatment of cancer and other diseases, no drugs targeting this target have been marketed so far. Among these drugs in clinical research, the clinically most important grade 3/4 and dose-limiting adverse side effect of BAY1251152 is neutropenia. The kinase selectivity and metabolism of AZD4573 are not good, which limits its effectiveness. Therefore, there is still an urgent need to develop novel, safer and more effective CDK9 inhibitors that can treat a variety of cancers (including leukemia and lymphoma).

### SUMMARY

In one aspect, the present invention provides a compound represented by formula (I), a pharmaceutically acceptable salt or an isomer thereof,

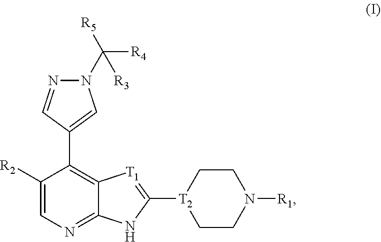

### Where, T₁ is N or CR;

### R is H or Cl;

### T₂ is N or CH;

R₁ is H or C₁₋₆ alkyl, where the C₁₋₆ alkyl is optionally substituted with 1, 2 or 3 substituents independently selected from F, Cl, —OH, —NH₂ and C₁₋₃ alkoxy;

### R₂ is H, F or Cl;

R₃ and R₄ are each independently H, F, Cl or C₁₋₃ alkyl;
R₅ is H, C₃₋₆ cycloalkyl or phenyl, where the C₃₋₆ cycloalkyl and phenyl are optionally substituted with 1, 2 or 3 Rₐ;
each Rₐ is independently H, F, Cl, C₁₋₃ alkyl or C₁₋₃ alkoxy.

In some aspects of the present invention, the above-mentioned compound has a structure represented by formula (I-1) or (I-2):

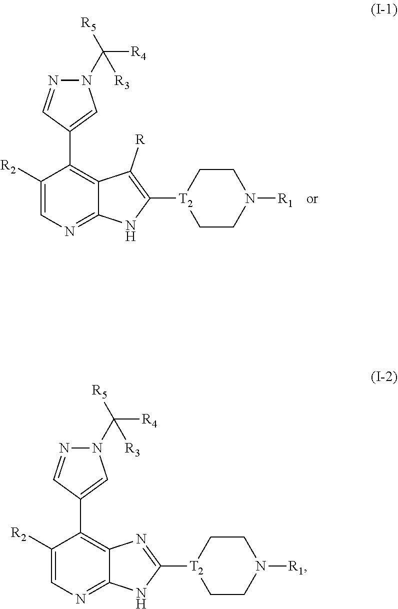

Where, R, T₂, R₁, R₂, R₃, R₄ and R₅ are as defined in the present invention.

In some aspects of the present invention, the above-mentioned compound has a structure represented by formula (I-1-a) or (I-1-b):

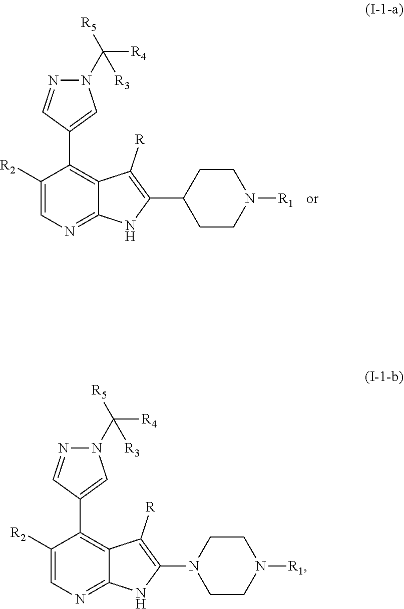

Where, R, R₁, R₂, R₃, R₄ and R₅ are as defined in the present invention.

In some aspects of the present invention, the above-mentioned R₃ and R₄ are each independently H, F or

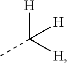

and other variables are as defined in the present invention.

In some aspects of the present invention, the above-mentioned R₃ and R₄ are each independently H, and other variables are as defined in the present invention.

In some aspects of the present invention, the above-mentioned compound has a structure represented by formula (I-1-c) or (I-1-d):

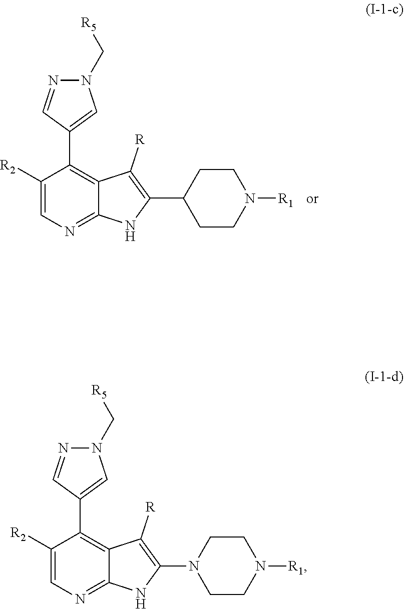

Where, R, R₁, R₂ and R₅ are as defined in the present invention.

In some aspects of the present invention, each of the above Rₐ is independently H, F, Cl or

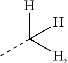

and other variables are as defined in the present invention.

In some aspects of the present invention, each of the above Rₐ is independently Cl, and other variables are as defined in the present invention.

In some aspects of the present invention, the above-mentioned R₅ is H,

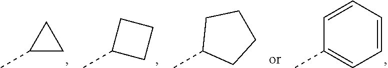

where said

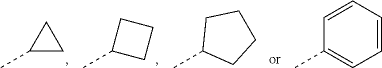

is optionally substituted with 1, 2 or 3 Rₐ, Rₐ and other variables are as defined in the present invention.

In some aspects of the present invention, the above-mentioned R₅ is H,

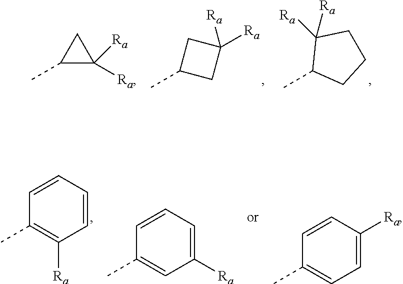

Rₐ and other variables are as defined in the present invention.

In some aspects of the present invention, the above-mentioned R₅ is H,

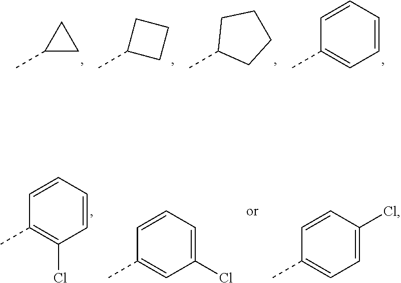

and other variables are as defined in the present invention.

In some aspects of the present invention, the above-mentioned R₅ is H,

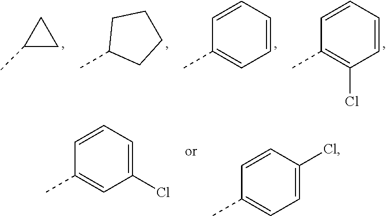

and other variables are as defined in the present invention.

In some aspects of the present invention, the above-mentioned compound has a structure represented by formula (I-1-e), (I-1-f), (I-1-g) or (I-1-h):

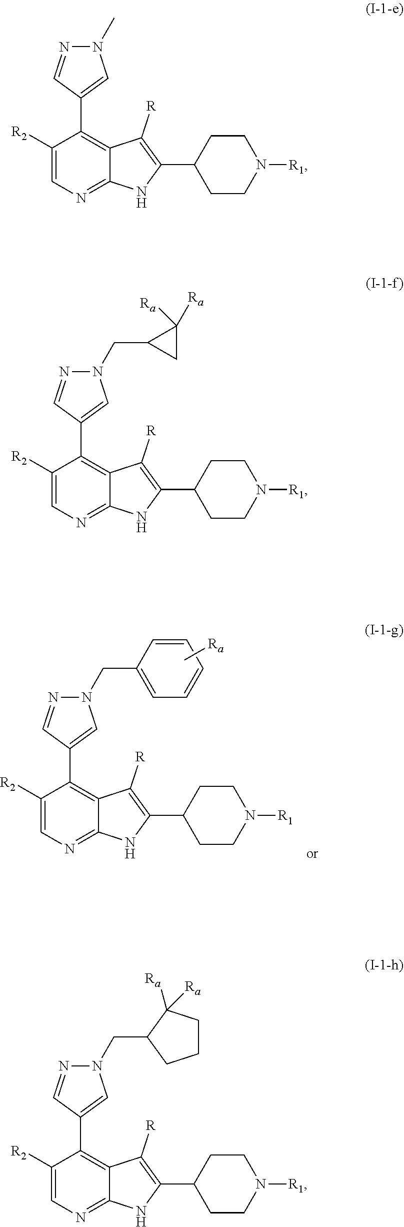

Where, R, R₁, R₂ and Rₐ are as defined in the present invention.

In some aspects of the present invention, the above-mentioned R₁ is H,

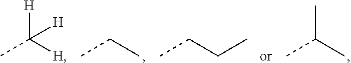

where the above-mentioned

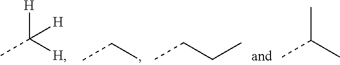

is optionally substituted with 1, 2 or 3 substituents independently selected from F, Cl, —OH, —NH₂ and —OCH₃, and other variables are as defined in the present invention.

In some aspects of the present invention, the above-mentioned R₁ is H,

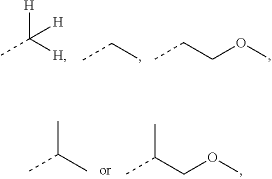

and other variables are as defined in the present invention.

In some aspects of the present invention, the above-mentioned structural units

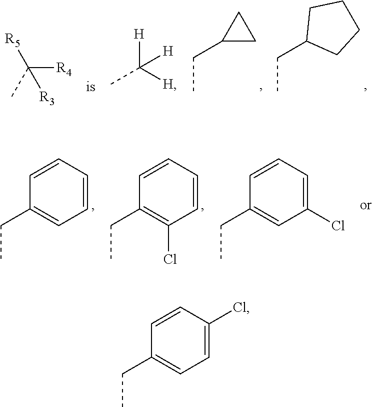

and other variables are as defined in the present invention.

There are also some schemes of the present invention that come from any combination of the above variables.

In some aspects of the present invention, the above-mentioned compound is:

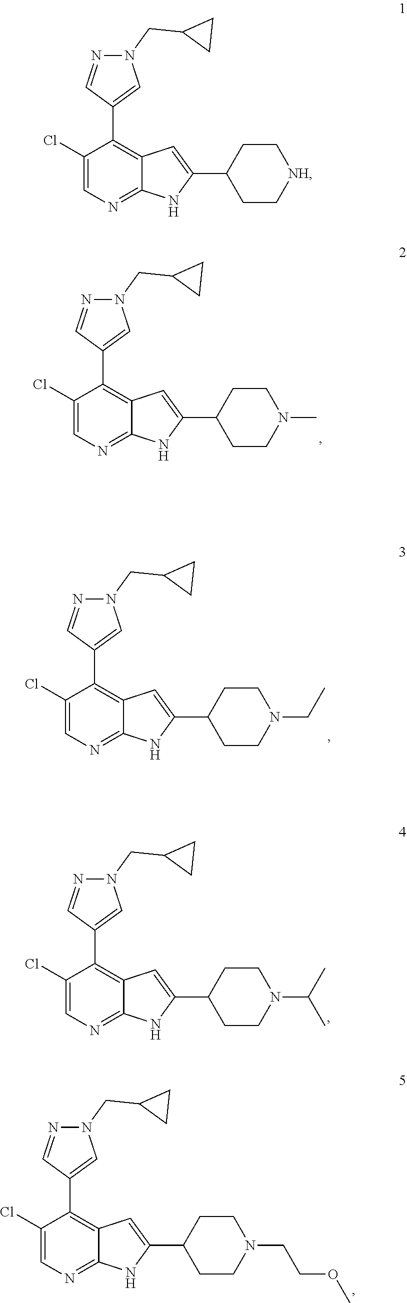 [SMILES: Clc1cnc2[nH]c(C3CCNCC3)cc2c1-c1cnn(CC2CC2)c1 ; CN1CCC(c2cc3c(-c4cnn(CC5CC5)c4)c(Cl)cnc3[nH]2)CC1 ; CCN1CCC(c2cc3c(-c4cnn(CC5CC5)c4)c(Cl)cnc3[nH]2)CC1 ; CC(C)N1CCC(c2cc3c(-c4cnn(CC5CC5)c4)c(Cl)cnc3[nH]2)CC1 ; COCCN1CCC(c2cc3c(-c4cnn(CC5CC5)c4)c(Cl)cnc3[nH]2)CC1]

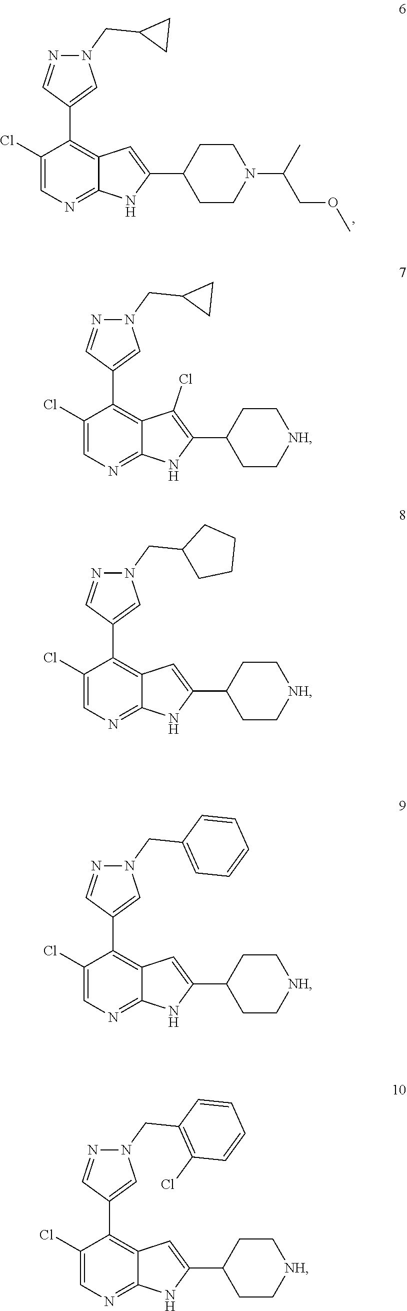 [SMILES: COCC(C)N1CCC(c2cc3c(-c4cnn(CC5CC5)c4)c(Cl)cnc3[nH]2)CC1 ; Clc1cnc2[nH]c(C3CCNCC3)c(Cl)c2c1-c1cnn(CC2CC2)c1 ; Clc1cnc2[nH]c(C3CCNCC3)cc2c1-c1cnn(CC2CCCC2)c1 ; Clc1cnc2[nH]c(C3CCNCC3)cc2c1-c1cnn(Cc2ccccc2)c1 ; Clc1ccccc1Cn1cc(-c2c(Cl)cnc3[nH]c(C4CCNCC4)cc23)cn1]

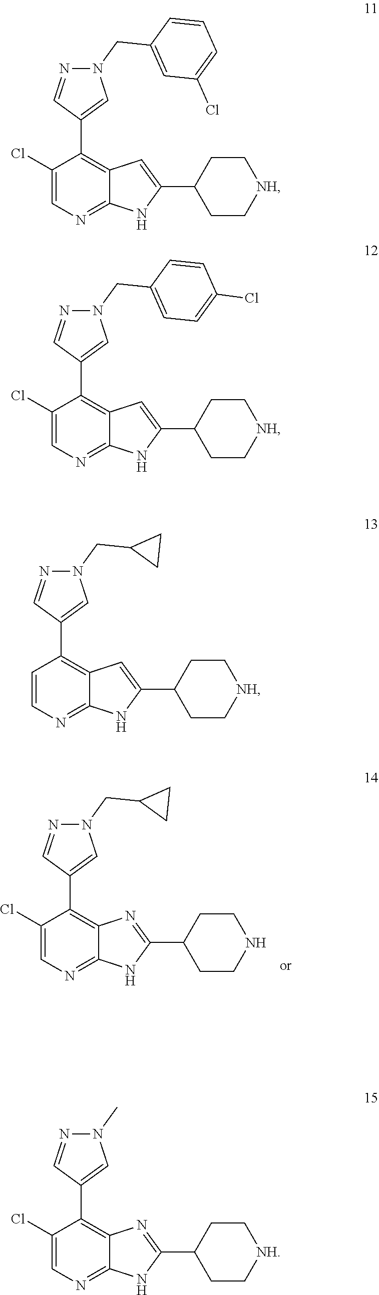 [SMILES: Clc1cccc(Cn2cc(-c3c(Cl)cnc4[nH]c(C5CCNCC5)cc34)cn2)c1 ; Clc1ccc(Cn2cc(-c3c(Cl)cnc4[nH]c(C5CCNCC5)cc34)cn2)cc1 ; c1cc(-c2cnn(CC3CC3)c2)c2cc(C3CCNCC3)[nH]c2n1 ; Clc1cnc2[nH]c(C3CCNCC3)nc2c1-c1cnn(CC2CC2)c1 ; Cn1cc(-c2c(Cl)cnc3[nH]c(C4CCNCC4)nc23)cn1]

The present invention also provides a pharmaceutical composition, which contains a therapeutically effective amount of the above-mentioned compound, a pharmaceutically acceptable salt or an isomer thereof, and a pharmaceutically acceptable carrier.

The present invention also provides the use of the above-mentioned compound, a pharmaceutically acceptable salt or an isomer thereof, and the above-mentioned pharmaceutical composition in the preparation of CDK9 inhibitor drugs.

The present invention also provides the use of the above-mentioned compound, a pharmaceutically acceptable salt or an isomer thereof, and the above-mentioned pharmaceutical composition in the preparation of a medicine for treating cancer.

The compound of the present invention is designed to have an azaindole structure as the core nucleus, which forms a strong double hydrogen bond with the hinge region of the CDK9 structure, and the nitrogen on the pyrazole forms a hydrogen bond with Lys48 of CDK9. In addition, the piperidine in the solvent region of the compound of the present invention is very basic. In CDK9, it can form a salt bridge with Asp109 and maintain high activity. In other subtypes of CDK such as CDK2, the positively charged piperidine has electrostatic repulsion with the positively charged Lys89 in the mouth of the cavity, so the molecule has better selectivity for CDK2 and other kinase subtypes. In mouse tumor models in vivo, the compound of the present invention has excellent anti-tumor activity and safety. It has a very good prospect for medicine.

### DETAILED DESCRIPTION

### Definition and Description

Unless otherwise stated, the following terms and phrases used herein are intended to have the following meanings. A specific term or phrase should not be considered uncertain or unclear unless specifically defined, but should be understood in its ordinary meaning. When a trade name appears herein, it is intended to refer to the corresponding commodity or an active ingredient thereof.

The term “pharmaceutically acceptable” as used herein refers to those compounds, materials, compositions and/or dosage forms, which are, within the scope of reliable medical judgment, suitable for use in contact with human and animal tissues, without excessive toxicity, irritation, allergic reactions or other problems or complications, and commensurate with a reasonable benefit/risk ratio.

The term “pharmaceutically acceptable salt” refers to a salt of the compound of the present invention, which is prepared from the compound with specific substituents discovered in the present invention with relatively non-toxic acids or bases. When the compound of the present invention contains a relatively acidic functional group, a base addition salt can be obtained by contacting the compound with a sufficient amount of base in a pure solution or a suitable inert solvent. The pharmaceutically acceptable base addition salt includes a salt of sodium, potassium, calcium, ammonium, organic amine or magnesium or similar salts. When the compound of the present invention contains a relatively basic functional group, the acid addition salt can be obtained by contacting the compound with a sufficient amount of acid in a pure solution or a suitable inert solvent. Examples of the pharmaceutically acceptable acid addition salt include an inorganic acid salt, where the inorganic acid includes, for example, hydrochloric acid, hydrobromic acid, nitric acid, carbonic acid, bicarbonate, phosphoric acid, monohydrogen phosphate, dihydrogen phosphate, sulfuric acid, hydrogen sulfate, hydroiodic acid, phosphorous acid, and the like; and an organic acid salt, where the organic acid includes, for example, acetic acid, propionic acid, isobutyric acid, maleic acid, malonic acid, benzoic acid, succinic acid, suberic acid, fumaric acid, lactic acid, mandelic acid, phthalic acid, benlenesulfonic acid, p-toluenesulfonic acid, citric acid, tartaric acid, and methanesulfonic acid, and the like; and an salt of amino acid (such as arginine and the like), and a salt of an organic acid such as glucuronic acid and the like. Certain specific compounds of the present disclosure that contain both basic and acidic functional groups can be converted to any base or acid addition salt.

The pharmaceutically acceptable salt of the present invention can be synthesized from the parent compound containing an acidic or basic moiety by conventional chemical methods. Generally, such salts are prepared by reacting these compounds in free acid or base form with stoichiometric amounts of appropriate bases or acid in water or organic solvents or a mixture of both.

The compound of the present invention may have a specific geometric or stereoisomeric form. The present invention contemplates all such compounds, including cis and trans isomers, (−)- and (+)-enantiomer, (R)- and (S)-enantiomer, diastereomer, (D)-isomer, (L)-isomer, and racemic mixtures and other mixtures, such as an enantiomer or diastereomer-enriched mixture, all of these mixtures are encompassed within the scope of the invention. The substituent such as alkyl can have an additional asymmetric carbon atom. All these isomers and mixtures thereof are included in the scope of the present invention.

Unless otherwise specified, the term “enantiomer” or “optical isomer” refers to stereoisomers that are mirror images of each other.

Unless otherwise specified, the term “cis-trans isomer” or “geometric isomer” is caused by the inability of a double bond or a single bond of carbon atoms on the ring to freely rotate.

Unless otherwise specified, the term “diastereomer” refers to a stereoisomer in which the molecule has two or more chiral centers and the relationship between the molecules is not mirror images.

Unless otherwise specified, “(+)” refers to dextrorotation, “(−)” refers to levorotation, and “(±)” refers to racemization.

Unless otherwise specified, using a wedged solid bond ( [symbol] ) and a wedged dashed bond ( [symbol] ) to represent the absolute configuration of a stereogenic center, using a straight solid bond ( [symbol] ) and a straight dashed bond ( [symbol] ) to represent the relative configuration of a stereogenic center. A wave line ( [symbol] ) represents a wedged solid bond ( [symbol] ) or a wedged dashed bond ( [symbol] ), or represents a straight solid bond ( [symbol] ) or a straight dashed bond ( [symbol] ).

The compound of the present invention may be present in particular. Unless otherwise specified, the term “tautomer” or “tautomeric form” refers to the fact that the isomers with different functional groups are in dynamic equilibrium at room temperature and can be converted into each other quickly. If tautomers are possible (such as in solution), the chemical equilibrium of the tautomers can be achieved. For example, proton tautomers (also known as prototropic tautomer) include interconversions by proton migration, such as keto-enol isomerization and imine-enamine isomerization. Valence tautomers include the mutual transformation caused by bonding electrons transfer. A specific example of keto-enol tautomerization is the interconversion between two tautomers pentane-2,4-dione and 4-hydroxypent-3-en-2-one.

Unless otherwise specified, the term “enriched in one isomer”. “isomer enriched”, “enriched in one enantiomer” or “enantiomer enriched” refers to the content of one of the isomers or enantiomers is less than 100%, and the content of this isomer or enantiomer is 60% or more, or 70% or more, or 80% or more, or 90% or more, or 95% or more, or 96% or more, or 97% or more, or 98% or more, or 99% or more, or 99.5% or more, or 99.6% or more, or 99.7% or more, or 99.8% or more, or 99.9% or more.

Unless otherwise specified, the term “excess of isomer” or “excess of enantiomer” refers to the difference between the relative percentages of two isomers or two enantiomers. For example, if the content of one of the isomers or enantiomers is 90%, and the content of the other is 10%, then the excess of isomer or enantiomer (ee value) is 80%.

The optically active (R)- and (S)-isomer and D and L isomer can be prepared by chiral synthesis or chiral reagents or other conventional techniques. If you want to obtain one kind of enantiomers of certain compound of the present invention, it can be prepared by asymmetric synthesis or derivative action of chiral auxiliary followed by separating the resulting diastereomeric mixture and cleaving the auxiliary group. Alternatively, when the molecule contains a basic functional group (such as amino) or an acidic functional group (such as carboxyl), the compound reacts with an appropriate optically active acid or base to form salts in the form of diastereomers which are then subjected to diastereomeric resolution through conventional methods in the art to give the pure enantiomer. In addition, the separation of enantiomers and diastereomers is usually accomplished through the use of chromatography, which employs a chiral stationary phase and is optionally combined with chemical derivatization (for example, carbamate generated from amine). The compound of the present invention may contain unnatural proportions of atomic isotope at one or more of the atoms constituting the compound. For example, compounds can be radiolabeled with a radioactive isotope, such as tritium (³H), iodine-125 (¹²⁵I) or C-14 (¹⁴C). For another example, deuterated drugs can be formed by replacing hydrogen with heavy hydrogen. The bond between deuterium and carbon is stronger than that of ordinary hydrogen and carbon. Compared with undeuterated drugs, deuterated drugs have advantages such as reduced side effects, increased drug stability, enhanced efficacy and prolonged biological half-life. All changes in the isotopic composition of the compounds of the present invention, whether radioactive or not, are included in the scope of the present invention. “Optional” or “optionally” means that the event or condition may occur but not requisite, and the description includes the instance in which the event or condition occurs and the instance in which the event or condition does not occur.

For drugs or pharmacologically active agents, the term “effective amount” or “therapeutically effective amount” refers to a sufficient amount of a drug or agent that is non-toxic but can achieve the desired effect. For the oral dosage form of the present invention, the “effective amount” of one active substance in the composition refers to the amount required to achieve the desired effect when combined with another active substance in the composition. The determination of the effective amount varies from person to person, depending on the age and general condition of the recipient, and also on the specific active substance. The appropriate effective amount in a case can be determined by those skilled in the art according to routine experiments.

The term “substituted” means that one or more hydrogen atoms on a specific atom are substituted with a substituent, and may include deuterium and hydrogen variants, as long as the valence of the specific atom is normal and the substituted compound is stable. When the substituent is an oxygen (i.e., ═O), it means that two hydrogen atoms are substituted. Oxygen substitution does not occur on aromatic groups. The term “optionally substituted” means that it can be substituted or unsubstituted. Unless otherwise specified, the type and number of substituents can be arbitrary on the basis that they can be chemically realized.

When any variable (such as R) occurs more than once in the composition or structure of a compound, its definition in each case is independent. Thus, for example, if a group is substituted with 0-2 R, the group can optionally be substituted with up to two R, and R has independent options in each case. In addition, a combination of substituent and/or variant thereof is only permitted only when the combination results in stable compounds.

When the number of a linking group is 0, such as —(CRR)₀—, indicates that the linking group is a single bond.

When one of the variables is selected from a single bond, it means that the two groups linked by the single bond are connected directly. For example, when L in A-L-Z represents a single bond, it means that the structure is actually A-Z.

When a substituent is vacant, it means that the substituent is absent. For example, when X is vacant in A-X, it means that the structure is actually A. When the listed substituents do not indicate by which atom is connected to the substituted group, such substituents can be bonded by any atom thereof. For example, when pyridyl acts as a substituent, it can be linked to the group to be substituted with any carbon atom on the pyridine ring.

When an enumerative linking group does not indicate its linking direction, the linking direction is arbitrary. For example, the linking group L in

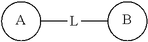

is -MW-. At this time, -MW- can connect ring A and ring B in the same direction as the reading order from left to right to form

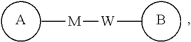

or read from left to right. Connect ring A and ring B in the opposite direction to form

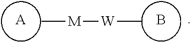

Combinations of the linking groups, substituents, and/or variants thereof are allowed only when such combination can result in a stable compound.

Unless otherwise specified, when a group has one or more connectable sites, any one or more sites of the group can be linked to other groups through chemical bonds. The chemical bond between the site and other groups can be represented by a straight solid bond ( [symbol] ), a strait dashed bond ( [symbol] ) or a wavy line

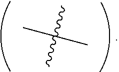

For example, the straight solid bond in —OCH₃ indicates that it is connected to other groups through the oxygen atom in the group; the straight dashed bonds in

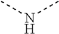

indicate that it is connected to other groups through the two ends of the nitrogen atom in the group; the wavy lines in

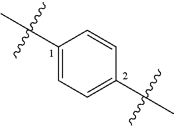 [SMILES: *c1ccc(*)cc1]

indicate that the phenyl group is connected to other groups through the 1 and 2 carbon atoms.

Unless otherwise specified, the number of atoms on the ring is usually defined as the number of ring members. For example, “5-7 membered ring” means that 5 to 7 atoms are arranged on the ring.

Unless otherwise specified, the term “C₁₋₆ alkyl” is used to represent a linear or branched saturated hydrocarbon group composed of 1 to 6 carbon atoms. The C₁₋₆ alkyl include C₁₋₅, C₁₋₄, C₁₋₃, C₁₋₂, C₂₋₆, C₂₋₄, C₆ and C₅ alkyl, etc.; it can be monovalent (such as methyl), bivalent (such as methylene) or multivalent (such as methine). Examples of C₁₋₆ alkyl include, but are not limited to, methyl (Me), ethyl (Et), propyl (including n-propyl and isopropyl), butyl (including n-butyl, isobutyl, s-butyl and t-butyl), pentyl (including n-pentyl, isopentyl and neopentyl), hexyl, etc.

Unless otherwise specified, the term “C₁₋₄ alkyl” is used to represent a linear or branched saturated hydrocarbon group composed of 1 to 4 carbon atoms. The C₁₋₄ alkyl include C₁₋₂, C₁₋₃ and C₂₋₃ alkyl, etc.; it can be monovalent (such as methyl), bivalent (such as methylene) or multivalent (such as methine). Examples of C₁₋₄ alkyl include, but are not limited to, methyl (Me), ethyl (Et), propyl (including n-propyl and isopropyl), butyl (including n-butyl, isobutyl, s-butyl and t-butyl), etc.

Unless otherwise specified, the term “C₁₋₃ alkyl” is used to represent a linear or branched saturated hydrocarbon group composed of 1 to 3 carbon atoms. The C₁₋₃ alkyl include C₁₋₂ and C₂₋₃ alkyl, etc.; it can be monovalent (such as methyl), bivalent (such as methylene) or multivalent (such as methine). Examples of C₁₋₃ alkyl include, but are not limited to, methyl (Me), ethyl (Et), propyl (including n-propyl and isopropyl), etc.

Unless otherwise specified, the term “C₁₋₃ alkoxy” means those alkyl consisting of 1 to 3 carbon atoms that are connected to the rest of the molecule through an oxygen atom. The C₁₋₃ alkoxy includes C₁₋₂, C₂₋₃, C₃ and C₂ alkoxy and so on. Examples of C₁₋₃ alkoxy include, but are not limited to, methoxy, ethoxy, propoxy (including n-propoxy and isopropoxy), etc.

Unless otherwise specified, “C₃₋₆ cycloalkyl” means a saturated cyclic hydrocarbon group consisting of 3 to 6 carbon atoms, which comprises a monocyclic and bicyclic ring system, the C₃₋₆ cycloalkyl includes C₃₋₅, C₃₋₄ or C₄₋₅ cycloalkyl, etc.; it can be monovalent, bivalent or multivalent. Examples of C₃₋₆ cycloalkyl include, but are not limited to, cyclopropyl, cyclobutyl, cyclopentyl, cyclohexyl, etc.

Unless otherwise specified, “C₃₋₅ cycloalkyl” means a saturated cyclic hydrocarbon group consisting of 3 to 5 carbon atoms, which comprises a monocyclic and bicyclic ring system, the C₃₋₅ cycloalkyl includes C₃₋₄ or C₄₋₅ cycloalkyl, etc.; it can be monovalent, bivalent or multivalent. Examples of C₃₋₅ cycloalkyl include, but are not limited to cyclopropyl, cyclobutyl, cyclopentyl, etc.

Unless otherwise specified, Cₙ₋ₙ₊ₘ or Cₙ-Cₙ₊ₘ includes any specific case of n to n+m carbons, such as C₁₋₁₂ including C₁, C₂, C₃, C₄, C₅, C₆, C₇, C₈, C₉, C₁₀, C₁₁, and C₁₂, also includes any range from n to n+m, such as C₁₋₁₂ including C₁₋₃, C₁₋₆, C₁₋₉, C₃₋₆, C₃₋₉, C₃₋₁₂, C₆₋₉, C₆₋₁₂, and C₉₋₁₂, etc.; in the same way, n-membered to n+m-membered means that the number of atoms in the ring is n to n+m. For example, a 3-12-membered ring includes a 3-membered ring, a 4-membered ring, a 5-membered ring, a 6-membered ring, a 7-membered ring, a 8-membered ring, a 9-membered ring, a 10-membered ring, a 11-membered ring, and a 12-membered ring, including any range from n to n+m, for example, 3-12 membered ring includes 3-6 membered ring, 3-9 membered ring, 5-6 membered ring, 5-7 membered ring, 6-7 membered ring, 6-8 membered ring, 6-10 membered ring, etc.

The term “leaving group” refers to a functional group or atom that can be substituted with another functional group or atom through a substitution reaction (for example, an affinity substitution reaction). For example, representative leaving groups include trifluoromethanesulfonate; chlorine, bromine, iodine; sulfonates, such as methanesulfonate, tosylate, p-bromobenzenesulfonate, p-toluenesulfonate, etc.; acyloxy, such as acetoxy, trifluoroacetoxy, etc.

The term “protecting group” includes, but is not limited to, “amino protecting group”, “hydroxy protecting group” or “thiol protecting group”. The term “amino protecting group” refers to a protecting group suitable for preventing side reactions at the amino nitrogen position. Representative amino protecting groups include but are not limited to: formyl; acyl, such as alkanoyl (e.g., acetyl, trichloroacetyl or trifluoroacetyl); alkoxycarbonyl, such as tert-butoxycarbonyl (Boc); aryl methoxycarbonyl, such as benzyloxycarbonyl (Cbz) and 9-fluorenylmethoxycarbonyl (Fmoc); aryl methyl, such as benzyl (Bn), triphenyl methyl (Tr), 1,1-bis-(4′-methoxyphenyl)methyl; silyl, such as trimethylsilyl (TMS) and tert-butyldimethylsilyl (TBS) and so on. The term “hydroxy protecting group” refers to a protecting group suitable for preventing side reactions of the hydroxyl group. Representative hydroxy protecting groups include, but are not limited to: alkyl, such as methyl, ethyl and tert-butyl; acyl, such as alkanoyl (e.g., acetyl); arylmethyl, such as benzyl (Bn), p-methoxybenzyl (PMB), 9-fluorenylmethyl (Fm) and diphenylmethyl (DPM); silyl, such as trimethylsilyl (TMS) and tert-butyldimethylsilyl (TBS), etc.

The compound of the present invention can be prepared by a variety of synthetic methods well known to those skilled in the art, including the specific embodiments listed below, the embodiments formed by their combination with other chemical synthesis methods, and equivalent alternative embodiments well known to a person skilled in the art, where the preferred embodiments include but are not limited to the examples of the present disclosure.

The solvents used in the present invention are commercially available.

The present invention uses the following abbreviations: DMF represents N,N-dimethylformamide; Cs₂CO₃ represents cesium carbonate; EtOAc represents ethyl acetate; EA represents ethyl acetate; THE represents tetrahydrofuran; MeOH represents methanol; DCM represents dichloromethane; PE represents petroleum ether; EtOH represents ethanol; CuI represents cuprous iodide; NCS represents N-chlorosuccinimide; NBS represents N-bromosuccinimide; ICI represents iodine monochloride; Pd(dppf)Cl₂ represents 1,1′-bis(diphenylphosphorus) ferrocene palladium chloride; Pd(PPh₃)₄ represents tetraphenylphosphine palladium; ACN represents acetonitrile; FA represents formic acid; NH₃.H₂O represents ammonia; TEA represents triethylamine; Boc₂O represents di-tert-butyl dicarbonate; represents Boc represents tert-butoxycarbonyl, which is a protecting group of amino group; CDI represents N,N′-carbonyl diimidazole: LCMS represents liquid mass spectrometry chromatography; HPLC represents liquid chromatography; TLC represents thin layer chromatography.

Compounds are named according to conventional naming principles in the field or ChemDraw® software, and commercially available compounds use supplier catalog names.

### EXAMPLES

The following examples describe the present invention in detail, but they are not meant to impose any unfavorable limitation on the present invention. The present invention has been described in detail herein, and its specific embodiments are also disclosed. For those skilled in the art, it will be obvious that various changes and improvements can be made to the embodiments of the present invention without departing from the spirit and scope of the present invention. The hydrochloride or formate of the compound of the present invention is added with saturated sodium bicarbonate solution to adjust the pH to neutral, and separated by high performance liquid chromatography (neutral, ammonium bicarbonate system) to obtain the free base of the compound.

### Reaction Scheme 1: Preparation of Compound Represented by Formula (I-1-a),

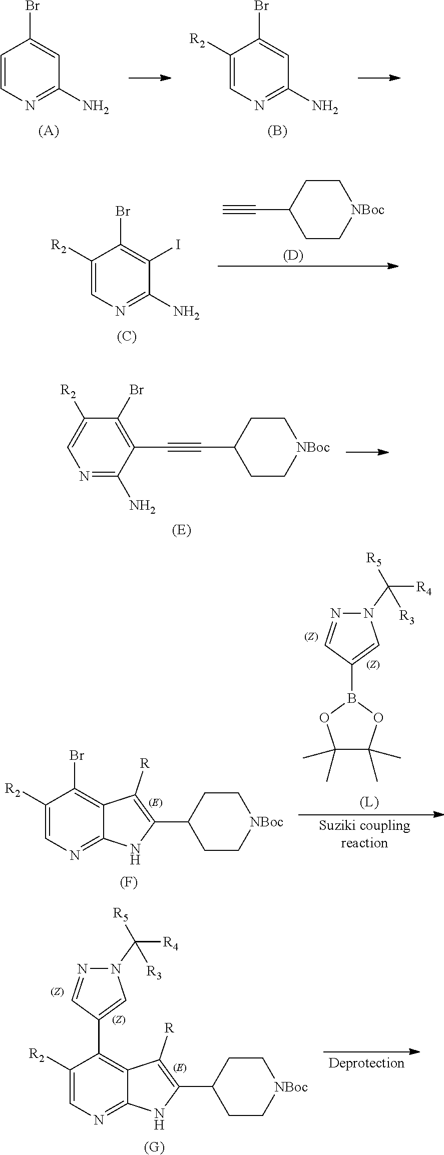

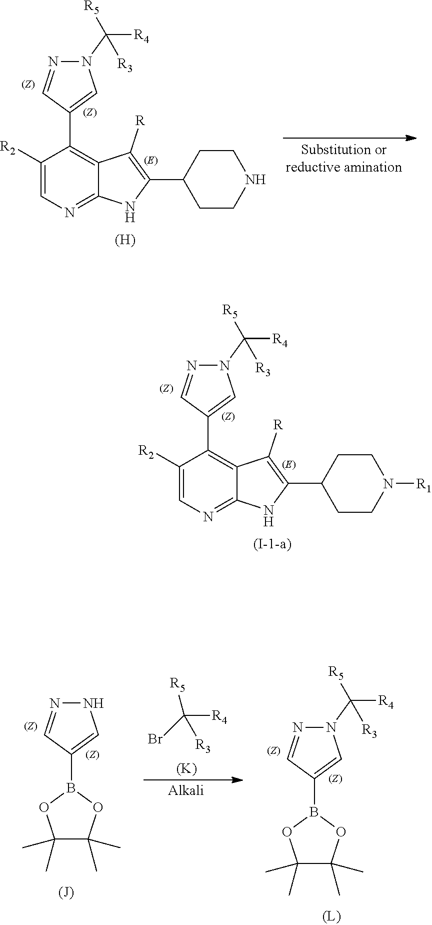

In the reaction shown in reaction scheme 1, R, R₁, R₂, R₃, R₄ and R₅ are as defined in the present invention, compound (E) can be prepared by sonigashira coupling reaction between compound (C) and compound (D). This reaction requires a suitable catalyst (such as tetrakistriphenyiphosphine palladium, cuprous iodide), a suitable base (such as triethylamine), a suitable solvent (such as toluene, acetonitrile). According to reaction scheme 1, compound (F) can be prepared by ring closure of compound (E). This reaction is more preferred to be carried out at high temperature and requires a suitable base (such as potassium tert-butoxide) and a suitable solvent (such as DMF). Compound (L) can be prepared by substitution reaction between compound (J) and bromide (K). This reaction is more preferred to proceed at high temperature and requires a suitable base (such as cesium carbonate) and a suitable solvent (such as DMF).

Compound (F) and compound (L) are catalyzed by palladium (such as Pd(dppt)Cl₂) under coupling to obtain compound (G). Compound (G) is deprotected under acidic conditions (such as hydrogen chloride/ethyl acetate solution) to obtain compound (H), and compound (H) undergoes substitution reaction with corresponding starting materials or reductive amination reaction to obtain the compound represented by formula (I-1-a).

### Example 1

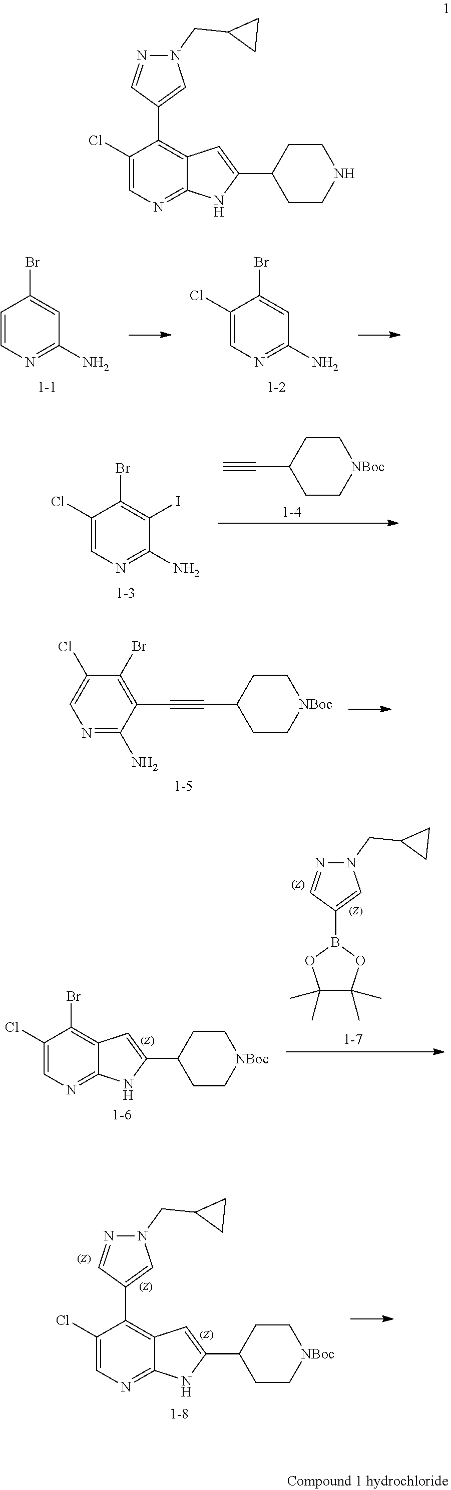

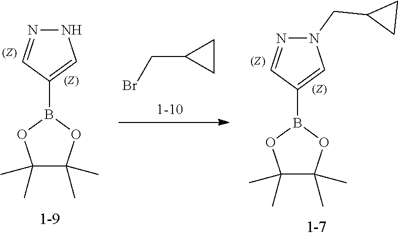

### Step 1:

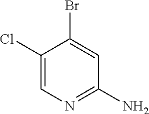

To solution of compound 1-1 (30.0 g, 173.40 mmol, 1.0 equivalent) in N,N-dimethylformamide (300) mL) was added N-chlorosuccinimide (27.79 g, 208.08 mmol, 1.2 equivalent) at −20° C. The mixture was reacted at 25° C. for 1 hour. TLC (petroleum ether:ethyl acetate=3:1) showed that the reaction of the starting material was complete. The reaction solution was poured into an aqueous solution of sodium hydroxide (w %=10%, 500 mL), extracted with ethyl acetate (300 mL*2), the organic phases were combined, washed with saturated brine (300 mL), and the organic phase was separated. Dry with water sodium sulfate, filter, and spin-dry the filtrate to obtain a residue. The residue was purified by silica gel column (eluent: petroleum ether:ethyl acetate=40:1 to 10:1) to obtain compound 1-2. LCMS (ESI) m/z: 208.9 (M+1).

### Step 2:

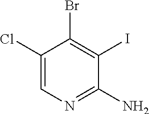

To solution of compound 1-2 (10.0 g, 48.20 mmol, 1.0 equivalent) in N,N-dimethylformamide (200 mL) was added iodine monochloride (11.74 g, 72.30 mmol, 3.69 mL, 1.5 equivalent) at 40° C., and the mixture was reacted at 40° C. for 3 hours. TLC (petroleum ether:ethyl acetate=3:1) showed that the starting material had not reacted completely. The reaction solution was allowed to continue to react at 40° C. for 12 hours. TLC (petroleum ether:ethyl acetate=3:1) showed that the reaction of the starting material was complete. The reaction solution was poured into water (600 mL), extracted with dichloromethane (500 mL), and the organic phase was washed sequentially with sodium sulfite (300 mL*2) and saturated brine (300 mL). The organic phase was concentrated to obtain a residue. The residue was purified by silica gel column (eluent:petroleum ether:ethyl acetate:=40:1 to 10:1) to obtain compound 1-3.

### Step 3:

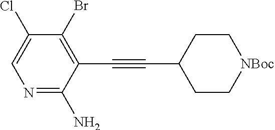 [SMILES: CC(C)(C)OC(=O)N1CCC(C#Cc2c(N)ncc(Cl)c2Br)CC1]

To solution of compound 1-3 (5.0 g, 15.00 mmol, 1.0 equivalent) in triethylamine (100 mL) was added compound 1-4 (4.71 g, 22.50 mmol, 1.50 equivalent), dichlorobis(triphenylphosphine)palladium (2.11 g, 3.00 mmol, 0.20 equivalent) and ketone iodide (2.86 g, 15.00 mmol, 1.0 equivalent), the reaction solution was replaced with nitrogen three times, and then reacted at 110° C. for 12 hours. LCMS showed that the starting material had not reacted completely. The reaction solution was cooled to 30° C., filtered through celite, and the filter cake was washed with ethyl acetate (50 mL). The filtrate was concentrated under reduced pressure, and the obtained residue was purified by silica gel column (petroleum ether:ethyl acetate=20:1 to 5:1) to obtain compound 1-5. LCMS (ESI) m/z: 415.5 (M+1).

### Step 4:

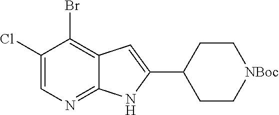 [SMILES: CC(C)(C)OC(=O)N1CCC(c2cc3c(Br)c(Cl)cnc3[nH]2)CC1]

To solution of compound 1-5 (2.0 g, 4.82 mmol, 1.0 equivalent) in N,N-dimethylformamide (100 mL) was added potassium tert-butoxide (1.62 g, 14.47 mmol, 3.0 equivalent), and after the reaction liquid was replaced with nitrogen three times, the reaction was carried out at 110° C. for 2 hours. TLC (petroleum ether:ethyl acetate=3:1) showed that the reaction was complete. The reaction solution was cooled to room temperature, filtered, and the filter cake was washed with ethyl acetate (50 mL). The filtrate was concentrated under reduced pressure, and the obtained residue was purified by silica gel column chromatography (petroleum ether:ethyl acetate:=10:1 to 3:1) to obtain compound 1-6. LCMS (ESI) m/z: 415.8 (M+1).

### Step 5:

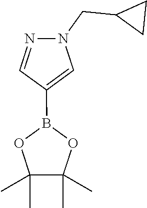 [SMILES: CC1(C)OB(c2cnn(CC3CC3)c2)OC1(C)C]

To solution of compound 1-9 (2.0 g, 10.31 mmol, 1.0 equivalent) in N,N-dimethylformamide (100 ml) was added bromomethylcyclopropane (1-10, 1.67 g, 12.37 mmol, 1.18 mL, 1.2 equivalent) and cesium carbonate (10.07 g, 30.92 mmol, 3.0 equivalent). The mixture was reacted at 80° C. for 5 hours, and TLC (petroleum ether:ethyl acetate=3:1) showed that the reaction of the starting material was complete. The reaction solution was cooled to 20° C., poured into water (60 mL), extracted with ethyl acetate (60 mL), and the organic phase was washed once with saturated brine (60 mL). The organic phase was separated and concentrated to obtain a residue. The residue was purified by silica gel column (petroleum ether:ethyl acetate=10:1 to 3:1) to obtain compound 1-7. LCMS (ESI) m/z: 249.2 (M+1).

### Step 6:

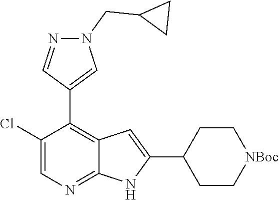 [SMILES: CC(C)(C)OC(=O)N1CCC(c2cc3c(-c4cnn(CC5CC5)c4)c(Cl)cnc3[nH]2)CC1]

To solution of compound 1-6 (200 mg, 482.25 μmol, 1.0 equivalent) in 1,4-dioxane (20 mL) and water (5 mL) was added compound 1-7 (358.98 mg, 1.45 mmol, 3.0 equivalent), Pd(dppf)Cl₂ (7.06 mg, 9.64 μmol, 0.2 equivalent) and cesium carbonate (471.38 mg, 1.45 mmol, 3.0 equivalent). After the reaction liquid was replaced with nitrogen three times, the reaction was carried out at 100° C. for 12 hours. LCMS showed that the reaction of the starting material was complete, the reaction solution was cooled to 20° C., and concentrated under reduced pressure to obtain a residue. The residue was purified by thin chromatography plate (petroleum ether:ethyl acetate=1:1) to obtain compound 1-8. LCMS (ESI) m/z: 456.2 (M+1).

### Step 7:

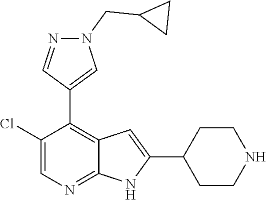 [SMILES: Clc1cnc2[nH]c(C3CCNCC3)cc2c1-c1cnn(CC2CC2)c1]

To solution of compound 1-8 (120 mg, 263.17 μmol, 1.0 equivalent) in ethyl acetate (5 mL) was added hydrogen chloride/ethyl acetate (4 mol/L, 5 mL, 76.0 equivalent). The mixture was reacted at 20° C. for 12 hours. LCMS showed that the reaction of the starting material was complete, and the reaction solution was concentrated to obtain a residue. The residue was purified by preparative HPLC (chromatographic column: Phenomenex Synergi C18 150*25 mm*10 μm, hydrochloric acid, mobile phase: water (0.05% hydrochloric acid)-acetonitrile, gradient: acetonitrile 13%-33%) to obtain the hydrochloride of compound 1. ¹H NMR (400 MHz, DMSO-d₆) δ 12.03 (s, 1H), 9.33 (br d, J=10.0 Hz, 11H), 9.11 (br d, J=9.8 Hz, 1H), 8.38 (s, 1H), 8.20 (s, 1H), 7.99 (s, 1H), 6.35 (d, J=1.3 Hz, 1H), 4.10 (d, J=7.2 Hz, 2H), 3.33 (br d, J=12.5 Hz, 2H), 3.13-2.95 (m, 3H), 2.24 (br d, J=12.5 Hz, 2H), 1.97-1.85 (m, 2H), 1.38-1.26 (m, 1H), 0.59-0.52 (m, 21H), 0.45-0.39 (m, 2H); LCMS (ESI) m/z: 356.2 (M+1).

### Example 2

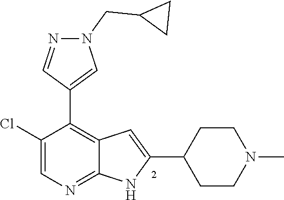 [SMILES: CN1CCC(c2cc3c(-c4cnn(CC5CC5)c4)c(Cl)cnc3[nH]2)CC1]

To solution of compound 1 (70 mg, 152.46 μmol, 1.0 equivalent) in methanol (2.0 mL) was added 37% formaldehyde (218 mg, 2.69 mmol, 0.2 mL, 17.85 equivalent) solution and sodium cyanoborohydride (94.55 mg, 1.50 mmol, 10.0 equivalent) at 20° C., and the mixture was stirred at 20° C. for 1 hour. LCMS showed that the reaction of starting material was complete. The pH of the reaction mixture was adjusted to 7 with 1 mol/L hydrochloric acid solution, and concentrated under reduced pressure, and the resulting residue was purified by preparative HPLC (chromatographic column: Phenomenex Synergi C18 150*25 mm*10 μm; formic acid, mobile phase: [water (0.225%) formic acid)-acetonitrile], gradient: acetonitrile 22%-52%) to obtain the formate of compound 2. ¹H NMR (400 MHz, DMSO-d₆) δ 11.82 (br s, 1H), 8.35 (s, 1H), 8.28 (br s, 1H), 8.16 (s, 1H), 7.98 (s, 1H), 6.33 (s, 1H), 4.09 (d, J=7.09 Hz, 2H), 3.04 (br d, J=10.88 Hz, 2H), 2.78 (br t, J=11.0) Hz, 1H), 2.39-2.28 (m, 5H), 2.05 (br d, J=11.98 Hz, 2H), 1.82 (q, J=11.41 Hz, 2H), 1.37-1.28 (m, 1H), 0.60-0.53 (m, 2H), 0.46-0.40 (m, 2H): LCMS (ESI) m/z: 370.0 (M+1).

### Example 3

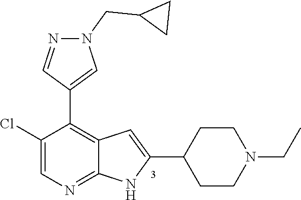 [SMILES: CCN1CCC(c2cc3c(-c4cnn(CC5CC5)c4)c(Cl)cnc3[nH]2)CC1]

To solution of compound 1 (150 mg, 228.30 μmol, 1.0 equivalent) in methanol (5 mL) was added acetaldehyde (125.71 mg, 1.14 mmol, 160.14 μL, 5.0 equivalent) and sodium cyanoborohydride (71.73 mg, 1.14 mmol, 5.0 equivalent). The mixture was reacted at 15° C. for 2 hours. LCMS showed that the reaction of the starting material was complete, and the reaction solution was concentrated to obtain a residue. Water (10 mL) and ethyl acetate (20 mL) were added to the residue, the organic phase was separated and concentrated to obtain a residue. The residue was purified by preparative HPLC (chromatographic column: Phenomenex Synergi C18 150*25 mm*10 μm; hydrochloric acid, mobile phase: [water (0.05% hydrochloric acid)-acetonitrile], gradient: acetonitrile 10%-40%) to obtain the hydrochloride of compound 3. ¹H NMR (400 MHz, DMSO-d₆) δ 12.13-11.86 (m, 1H), 10.68 (br s, 1H), 8.44-8.34 (m, 1H), 8.20 (s, 1H), 8.06-7.92 (m, 1H), 6.53-6.29 (m, 1H), 4.10 (d, J=7.2 Hz, 2H), 3.55 (br d, J=11.4 Hz, 2H), 3.21-2.94 (m, 5H), 2.38-2.25 (m, 2H), 2.18-1.94 (m, 2H), 1.40-1.20 (m, 4H), 0.63-0.51 (m, 2H), 0.4840.37 (m, 2H); LCMS (ESI) m/z: 384.2 (M+1).

### Example 4

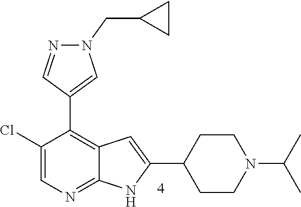 [SMILES: CC(C)N1CCC(c2cc3c(-c4cnn(CC5CC5)c4)c(Cl)cnc3[nH]2)CC1]

To solution of compound 1 (100 mg, 152.20 μmol, 1.0 equivalent) in methanol (2 mL) was added acetone (2.63 g, 45.34 mmol, 3.33 mL, 297.90 equivalent) and sodium cyanoborohydride (95.64 mg, 1.52 mmol, 10.0 equivalent) at 20° C., and the mixture was stirred at 20° C. for 2 hours. LCMS showed that the starting material remained. Acetone (2.63 g, 45.34 mmol, 3.33 mL, 297.90 equivalent) and sodium cyanoborohydride (95.64 mg, 1.52 mmol, 10 equivalent) were continuously added, and the mixture was stirred at 20° C. for 12 hours. LCMS showed that the reaction of the starting material was complete. The reaction mixture was concentrated under reduced pressure, dissolved in water (10 mL), and extracted with ethyl acetate (20 mL*2). The organic phase was dried over anhydrous sodium sulfate and concentrated under reduced pressure to obtain a crude product. The crude product was purified by preparative HPLC (chromatographic column: Phenomenex Synergi C18 150*25 mm*10 μm; hydrochloric acid, mobile phase: water (0.05% hydrochloric acid)-acetonitrile, gradient: acetonitrile 11%-41%) to obtain the hydrochloride of compound 4. ¹H NMR (400 MHz, DMSO-d₆) δ 12.08-11.92 (m, 1H), 10.54 (br s, 1H), 8.42-8.34 (m, 1H), 8.21 (s, 1H), 7.98 (s, 1H), 6.33 (d, J=1.10 Hz, 1H), 4.10 (d, J=7.21 Hz, 2H), 3.45 (br d, J=11.13 Hz, 3H), 3.16-3.03 (m, 3H), 2.34 (br d, J=11.13 Hz, 2H), 2.21-2.02 (m, 2H), 1.36-1.23 (m, 7H), 0.62-0.53 (m, 2H), 0.62-0.53 (m, 2H); LCMS (EST) m/z: 398.2 (M+1).

### Example 5

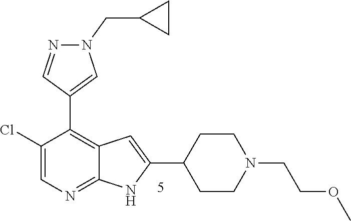 [SMILES: COCCN1CCC(c2cc3c(-c4cnn(CC5CC5)c4)c(Cl)cnc3[nH]2)CC1]

To solution of compound 1 (200 mg, 304.40 μmol, 1.0 equivalent) in N,N-dimethylformamide (10 mL) was added 1-bromo-2-methoxyethane (211.54 mg, 1.52 mmol, 142.93 μL, 5.0 equivalent) and triethylamine (154.01 mg, 1.52 mmol, 211.84 μL, 5.0 equivalent). The mixture was reacted at 15° C. for 12 hours. LCMS showed that the reaction of the starting material was complete, and the reaction solution was concentrated to obtain a residue. The residue was purified by preparative HPLC (chromatographic column: Phenomenex Synergi C18 150*25 mm*10 μm; hydrochloric acid, mobile phase: water (0.05% hydrochloric acid)-acetonitrile, gradient: acetonitrile 10%-40%) to obtain the hydrochloride of compound 5. ¹H NMR (400 MHz, DMSO-d₆) δ 12.31-12.08 (m, 1H), 10.95 (br s, 1H), 8.48-8.35 (m, 1H), 8.23 (s, 1H), 8.07-7.96 (m, 1H), 6.66-6.26 (m, 1H), 4.10 (d, J=7.2 Hz, 2H), 3.85-3.68 (m, 2H), 3.63-3.38 (m, 2H), 3.33-3.23 (m, 5H), 3.22-2.98 (m, 3H), 2.30 (br d, J=13.8 Hz, 2H), 2.21-1.98 (m, 2H), 1.41-1.22 (m, 1H), 0.59-0.50 (m, 2H), 0.46-0.37 (m, 2H). LCMS (ESI) m/z 414.2 (M+1).

### Example 6

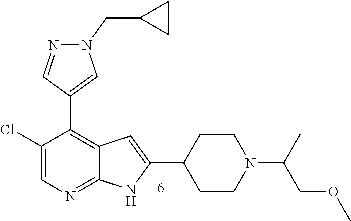 [SMILES: COCC(C)N1CCC(c2cc3c(-c4cnn(CC5CC5)c4)c(Cl)cnc3[nH]2)CC1]

To solution of compound 1 (200 mg, 304.40 μmol, 1.0 equivalent) in methanol (10 mL) was added methoxyacetone (268.19 mg, 3.04 nmol, 282.30 μL, 10 equivalent) and cyano group sodium borohydride (191.29 mg, 3.04 mmol, 10 equivalent) at 20° C. and the mixture was stirred at 45° C. for 4 hours. LCMS showed that the starting material remained. The mixture was continuously stirred at 45° C. for 16 hours. LCMS showed that the reaction of the starting material was complete. The pH of the reaction solution was adjusted to 7 with 1 mol/L hydrochloric acid solution and concentrated under reduced pressure to obtain the residue by preparative HPLC (chromatographic column: Phenomenex Synergi C18 150*25 mm*10 μm; formic acid. [eluent: water (0.225% Formic acid)-acetonitrile], gradient: acetonitrile: 25%-55%) to obtain the formate of compound 6. ¹H NMR (400 MHz, DMSO-d₆) δ 11.79 (s, 1H), 8.35 (s, 1H), 8.23 (s, 1H), 8.15 (s, 1H), 7.98 (s, 1H), 6.32 (d, J=1.22 Hz, 1H), 4.09 (d, J=7.21 Hz, 2H), 3.51-3.44 (m, 1H), 3.33 (dd, J=9.66, 5.14 Hz, 1H), 3.29-3.22 (m, 3H), 3.06-2.92 (m, 3H), 2.82-2.71 (m, 1H), 2.64-2.54 (m, 2H), 2.04 (br d, J=11.98 Hz, 2H), 1.85-1.70 (m, 1H), 1.39-1.28 (m, 1H)), 1.04 (d, J=6.60 Hz, 31H), 0.59-0.51 (m, 2H), 0.45-0.38 (m, 2H); LCMS (ESI) m/z: 428.1 (M+1).

### Example 7

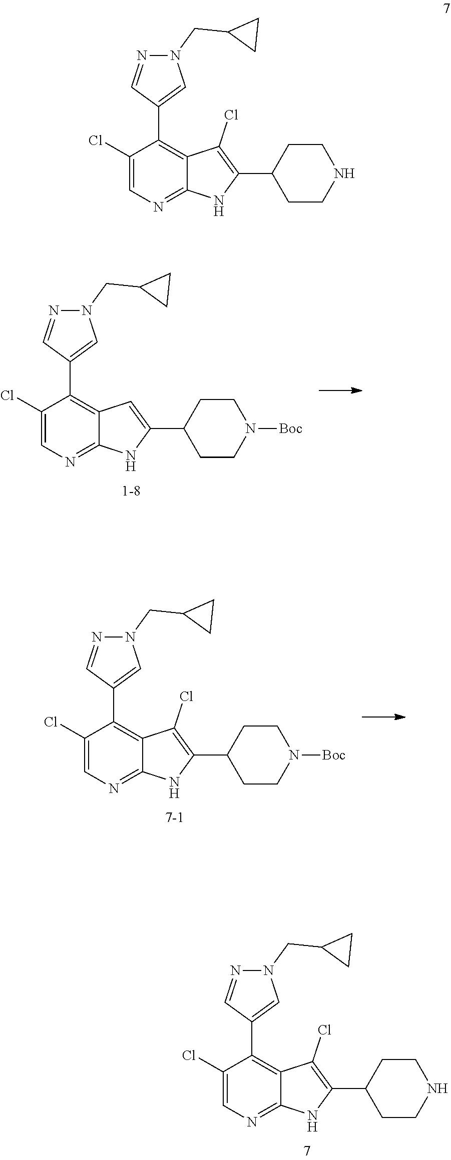 [SMILES: Clc1cnc2[nH]c(C3CCNCC3)c(Cl)c2c1-c1cnn(CC2CC2)c1 ; CC(C)(C)OC(=O)N1CCC(c2cc3c(-c4cnn(CC5CC5)c4)c(Cl)cnc3[nH]2)CC1 ; CC(C)(C)OC(=O)N1CCC(c2[nH]c3ncc(Cl)c(-c4cnn(CC5CC5)c4)c3c2Cl)CC1 ; Clc1cnc2[nH]c(C3CCNCC3)c(Cl)c2c1-c1cnn(CC2CC2)c1]

### Step 1:

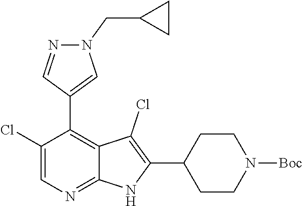 [SMILES: CC(C)(C)OC(=O)N1CCC(c2[nH]c3ncc(Cl)c(-c4cnn(CC5CC5)c4)c3c2Cl)CC1]

To solution of compound 1-8 (300 mg, 657.92 μmol, 1.0 equivalent) in N,N-dimethylformamide (10 mL) was added N-chlorosuccinimide (92.25 mg), 690.82 μmol, 282.30 μL, 1.05 equivalent) at 25° C., and the mixture was stirred at 25° C. for 2 hours. LCMS showed that the starting material remained, and the mixture was continuously stirred at 25° C. for 1 hour. LCMS showed that the reaction of the starting material was complete. The reaction solution was poured into water (30 mL) and filtered. The filter cake was washed with water (30 mL) and dissolved with ethyl acetate (30 mL). The organic phase was washed with brine (20 mL) and concentrated under reduced pressure to obtain a crude product. The crude product was purified by TLC (petroleum ether:ethyl acetate=2:1) plate to obtain compound 7-1. LCMS (ESI) m/z: 490.1 (M+1).

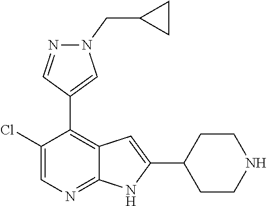 [SMILES: Clc1cnc2[nH]c(C3CCNCC3)cc2c1-c1cnn(CC2CC2)c1]

To solution of compound 7-1 (90 mg, 183.51 μmol, 1.0 equivalent) in ethyl acetate (5 mL) was added a hydrogen chloride/ethyl acetate solution (4.0 mol/L, 5 mL, 108.98 equivalent), and the mixture was reacted at 15° C. for 1 hour. LCMS showed that the reaction of the starting material was complete. The reaction solution was concentrated under reduced pressure, dissolved in water (10 mL), the pH of the aqueous phase was adjusted to 9 with saturated sodium carbonate, extracted with ethyl acetate (20 mL*2), the organic phase was washed with brine (20 mL), and concentrated under reduced pressure. The obtained residue was purified by preparative HPLC (chromatographic column: Phenomenex Synergi C18 150*25 mm*10 μm; formic acid, eluent: [water (0.225% formic acid)-acetonitrile], gradient: acetonitrile 9%-39%) to obtain compound 7. ¹H NMR (400 MHz, DMSO-d₆) δ 8.39 (s, 1H), 8.29 (s, 1H), 7.99 (s, 1H), 7.60 (s, 1H), 4.07 (d, J=7.0 Hz, 2H), 3.29-3.15 (m, 2H), 3.31-3.14 (m, 1H), 3.31-3.14 (m, 1H), 2.94-2.84 (m, 2H), 2.14-2.03 (m, 2H), 1.82 (br d, J=12.9 Hz, 2H), 1.34-1.25 (m, 1H), 0.57-0.51 (m, 2H), 0.42-0.37 (m, 2H): LCMS (ESI) m/z: 390.0 (M+1).

### Example 8

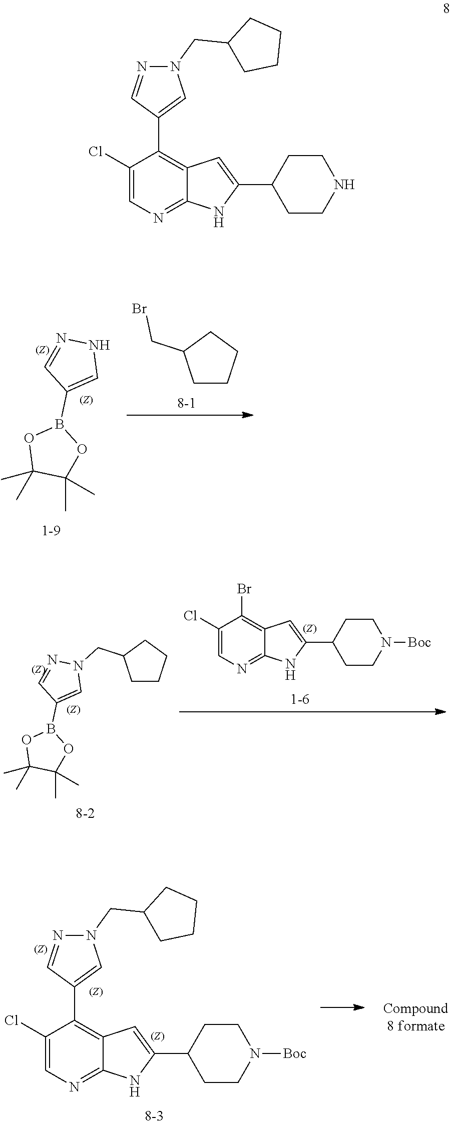 [SMILES: Clc1cnc2[nH]c(C3CCNCC3)cc2c1-c1cnn(CC2CCCC2)c1 ; CC1(C)OB(c2cn[nH]c2)OC1(C)C ; BrCC1CCCC1 ; CC1(C)OB(c2cnn(CC3CCCC3)c2)OC1(C)C ; CC(C)(C)OC(=O)N1CCC(c2cc3c(Br)c(Cl)cnc3[nH]2)CC1 ; CC(C)(C)OC(=O)N1CCC(c2cc3c(-c4cnn(CC5CCCC5)c4)c(Cl)cnc3[nH]2)CC1]

### Step 1:

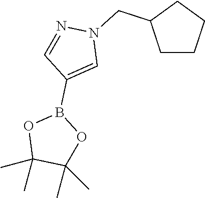 [SMILES: CC1(C)OB(c2cnn(CC3CCCC3)c2)OC1(C)C]

To solution of 1-9 (200 mg, 1.03 mmol, 1.0 equivalent) and 8-1 (201.68 mg, 1.24 mmol, 1.2 equivalent) in N,N-dimethylformamide (2 mL) was added cesium carbonate (1.01 g, 3.09 mmol, 3 equivalent) at 25° C., and the mixture was stirred at 100° C. for 12 hours. LCMS showed that the reaction of the starting material was complete. The reaction solution was poured into water (10 mL), extracted with ethyl acetate (20*2 mL), the organic phase was concentrated under reduced pressure, and the obtained residue was purified by silica gel column (petroleum ether:ethyl acetate=1:1) to obtain compound 8-2. LCMS (ESI) m/z: 277.1 (M+1).

### Step 2:

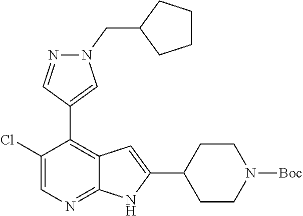 [SMILES: CC(C)(C)OC(=O)N1CCC(c2cc3c(-c4cnn(CC5CCCC5)c4)c(Cl)cnc3[nH]2)CC1]

To mixture of 1-6 (100 mg, 241.12 μmol, 1.0 equivalent) and 8-2 (98.89 mg, 361.69 μmol, 1.5 equivalent) in dioxane (2 mL) and water (0.5 mL) was added Pd(dppf)Cl₂ (17.64 mg, 24.11 μmol, 0.1 equivalent) and cesium carbonate (235.69 mg, 723.37 μmol, 3 equivalent) under the protection of nitrogen at 25° C., and the mixture was stirred at 100° C. for 12 hours. LCMS showed that the reaction of the starting material was complete. The reaction solution was filtered with celite, the filter cake was washed with ethyl acetate (30 mL), the filtrate was concentrated under reduced pressure, and the resulting residue was purified by TLC plate (petroleum ether:ethyl acetate=1:1) to obtain compound 8-3. LCMS (ESI) m/z: 484.3 (M+1).

### Step 3:

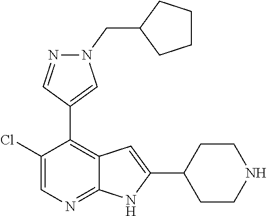 [SMILES: Clc1cnc2[nH]c(C3CCNCC3)cc2c1-c1cnn(CC2CCCC2)c1]

To solution of compound 8-3 (80 mg, 165.28 μmol, 1.0 equivalent) in ethyl acetate (5 mL) was added hydrogen chloride/ethyl acetate (4.0 mol/L, 5 mL, 121.02 equivalent), and the mixture was reacted at 15° C. for 1 hour. LCMS showed that the starting material remained, and the mixture was allowed to continue to react at 15° C. for 1 hour. LCMS showed that the reaction of the starting material was complete. The reaction solution was concentrated under reduced pressure, dissolved in water (10 mL), the pH of the aqueous phase was adjusted to 8 with saturated sodium bicarbonate, and extracted with ethyl acetate (20 mL*2), the organic phase was concentrated under reduced pressure, and the resulting residue was purified by preparative HPLC (chromatographic column: Boston Green ODS 150*30 mm*5 μm; formic acid, mobile phase: [water (0.225% formic acid)-acetonitrile], gradient: acetonitrile 15%-45%) to obtain the formate of compound 8. ¹H NMR (400 MHz, DMSO-d₄) δ 11.94 (br s, 11H), 8.44 (s, 1H), 8.46-8.39 (m, 1H), 8.34 (s, 11H), 8.17 (s, 1H), 7.97 (s, 1H), 6.31 (s, 1H), 4.15 (s, 2H), 3.26 (br d, J=11.1 Hz, 2H), 3.06-2.82 (m, 3H), 2.46-2.37 (m, 1H), 2.13 (br d, J=12.6 Hz, 2H), 1.83 (q, J=11.3 Hz, 2H), 1.69-1.47 (m, 6H), 1.37-1.22 (m, 2H): LCMS (ESI) m/z: 384.2 (M+1).

### Example 9

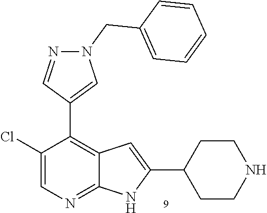 [SMILES: Clc1cnc2[nH]c(C3CCNCC3)cc2c1-c1cnn(Cc2ccccc2)c1]

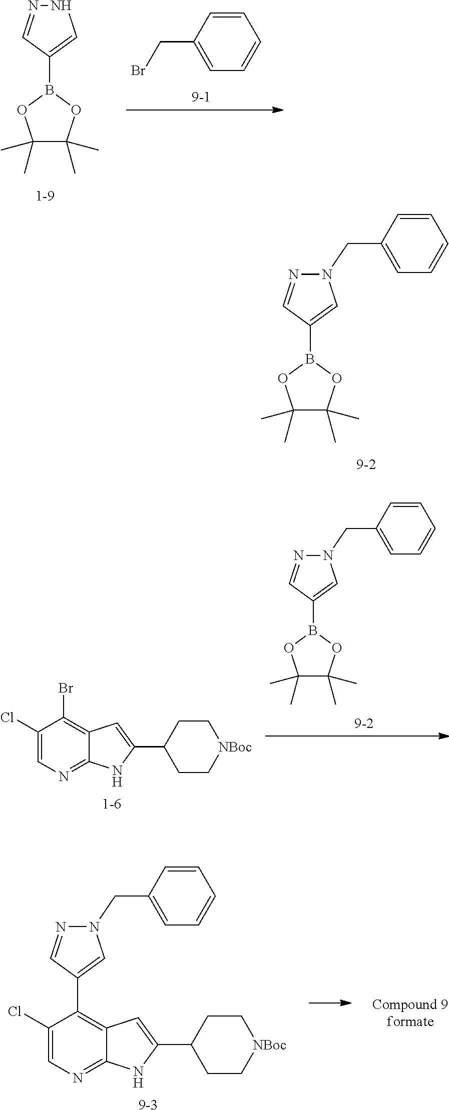

### Step 1:

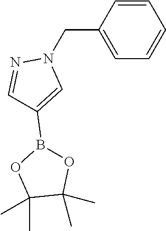 [SMILES: CC1(C)OB(c2cnn(Cc3ccccc3)c2)OC1(C)C]

To solution of compound 1-9 (1.0 g, 5.15 mmol, 1.0 equivalent) in N,N-dimethylformamide (30 mL) was added compound 9-1 (1.06 g, 6.18 mmol, 734.02 μL, 1.2 equivalent) and cesium carbonate (2.52 g, 7.73 mmol, 1.5 equivalent). The mixture was reacted at 100° C. for 12 hours. LCMS showed that the reaction of the starting material was complete. After the reaction solution was cooled to 15° C., it was poured into water (100 mL), the mixture was extracted with ethyl acetate (50 mL*2), the organic phases were combined, and concentrated under reduced pressure to obtain a residue. The residue was purified by silica gel column chromatography (eluent: petroleum ether:ethyl acetate=1:0 to 5:1) to obtain compound 9-2. LCMS (ESI) m/z: 284.9 (M+1).

### Step 2:

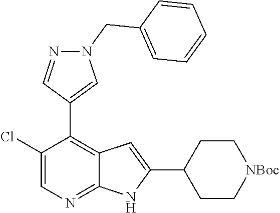 [SMILES: CC(C)(C)OC(=O)N1CCC(c2cc3c(-c4cnn(Cc5ccccc5)c4)c(Cl)cnc3[nH]2)CC1]

To solution of compound 1-6 (300 mg, 723.37 μmol, 1.0 equivalent) in 1,4-dioxane (20 mL) and water (10 mL) was added compound 9-2 (308.33 mg, 1.0) mmol, 1.5 equivalent), Pd(dppf)Cl₂ (105.86 mg, 144.67 μmol, 0.2 equivalent) and cesium carbonate (471.38 mg, 1.45 mmol, 2.0 equivalent). After the mixture was replaced with nitrogen three times, it was reacted at 100° C. for 2 hours. LCMS showed that the reaction of the starting material was complete. After the reaction solution was cooled to 15° C., it was concentrated to obtain a residue, and the residue was purified by a silica gel plate (petroleum ether:ethyl acetate=1:1) to obtain compound 9-3. LCMS (ESI) m/z: 492.2 (M+1).

### Step 3:

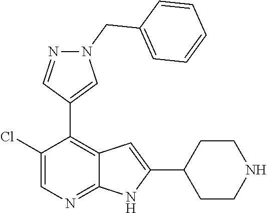 [SMILES: Clc1cnc2[nH]c(C3CCNCC3)cc2c1-c1cnn(Cc2ccccc2)c1]

To solution of compound 9-3 (170 mg, 320.23 μmol, 1.0 equivalent) in ethyl acetate (10 mL) was added a hydrogen chloride/ethyl acetate solution (4 mol/L, 10.0 mL, 124.91 equivalent). The mixture was allowed to continue to react at 15° C. for 2 hours, and LCMS showed that the reaction of the starting material was complete. The reaction solution was concentrated to obtain a residue. The residue was dissolved in water (20 mL). The pH was adjusted to ˜8 with an aqueous solution of sodium bicarbonate (w %=10%), and the mixture was extracted with dichloromethane/methanol=10:1 (50 mL). The organic phase was concentrated under reduced pressure to obtain a residue. The residue was purified by preparative HPLC (chromatographic column: Phenomenex Synergi CIS 150*25 mm*10 μm; formic acid, mobile phase: [water (0.225% formic acid)-acetonitrile], gradient: acetonitrile 11%-41%) to obtain the formate of compound 9. ¹H NMR (400 MHz, DMSO-d₆) δ 11.91 (br s, 1H), 8.49 (s, 1H), 8.42 (s, 114), 8.17 (s, 1H), 8.01 (s, 1H), 7.44-7.26 (m, 5H), 6.31 (s, 1H), 5.46 (s, 2H), 3.24 (br d, J=12.3 Hz, 2H), 3.05-2.80 (m, 3H), 2.12 (br d, J=12.2 Hz, 2H), 1.91-1.72 (m, 2H); LCMS (ESI) m/z: 392.1 (M+1).

### Example 10

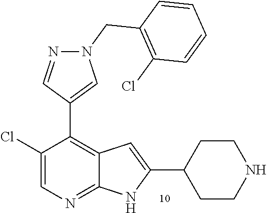 [SMILES: Clc1ccccc1Cn1cc(-c2c(Cl)cnc3[nH]c(C4CCNCC4)cc23)cn1]

For the synthesis of the formate of compound 10, refer to the formate of compound 9. ¹H NMR (400 MHz, DMSO-d₆) δ 11.97 (br s, 1H), 9.14 (br s, 1H), 8.88 (br d, J=9.2 Hz, 1H), 8.50 (s, 1H), 8.20 (s, 1H), 8.05 (s, 1H), 7.52 (br d, J=5.1 Hz, 1H), 7.37 (br d, J=4.8 Hz, 2H), 7.14 (br d, J=4.6 Hz, 1H), 6.33 (br s, 1H), 5.58 (br s, 2H), 3.34 (br d, J=11.4 Hz, 2H), 3.16-2.92 (m, 3H), 2.24 (br d, J=13.4 Hz, 2H), 1.87 (q, J=12.5 Hz, 2H); LCMS (ESI) m/z: 426.1 (M+1).

### Example 11

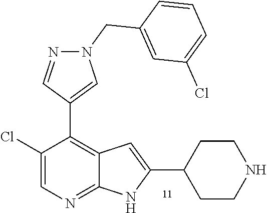 [SMILES: Clc1cccc(Cn2cc(-c3c(Cl)cnc4[nH]c(C5CCNCC5)cc34)cn2)c1]

For the synthesis of the formate of compound 11, refer to the formate of compound 9. ¹H NMR (400 MHz, DMSO-d₆) δ 11.98 (br s, 1H), 9.19 (br d, J=8.4 Hz, 1H), 8.93 (br d, J=8.8 Hz, 1H), 8.56 (s, 1H), 8.20 (s, 11H), 8.04 (s, 1H), 7.40 (br s, 3H), 7.30 (br d, J=6.9 Hz, 1H), 6.34 (br s, 1H), 5.49 (br s, 2H), 3.33 (br d, J=11.7 Hz, 2H), 3.18-2.91 (m, 3H), 2.24 (br d, J=13.4 Hz, 2H), 1.88 (q, J=12.3 Hz, 2H); LCMS (ESI) m/z: 426.1 (M+1).

### Example 12

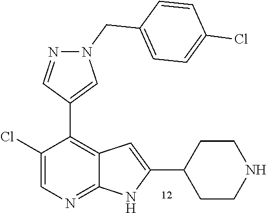 [SMILES: Clc1ccc(Cn2cc(-c3c(Cl)cnc4[nH]c(C5CCNCC5)cc34)cn2)cc1]

For the synthesis of compound 12, refer to compound 9. ¹H NMR (400 MHz, CD₃OD) δ 8.57-8.48 (m, 1H), 8.45-8.35 (m, 1H), 8.19-8.10 (m, 1H), 7.44-7.38 (m, 2H), 7.37-7.30 (m, 2H), 6.65-6.56 (m, 1H), 5.50 (s, 2H), 3.54 (br d, J=13.0 Hz, 2H), 3.27-3.11 (m, 3H), 2.36 (br d, J=12.5 Hz, 2H), 2.09-1.93 (m, 2H); LCMS (ESI) m/z: 426.1 (M+1).

### Example 13

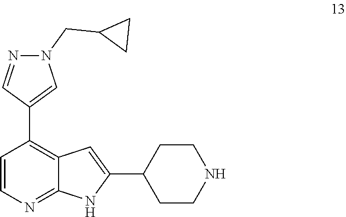 [SMILES: c1cc(-c2cnn(CC3CC3)c2)c2cc(C3CCNCC3)[nH]c2n1]

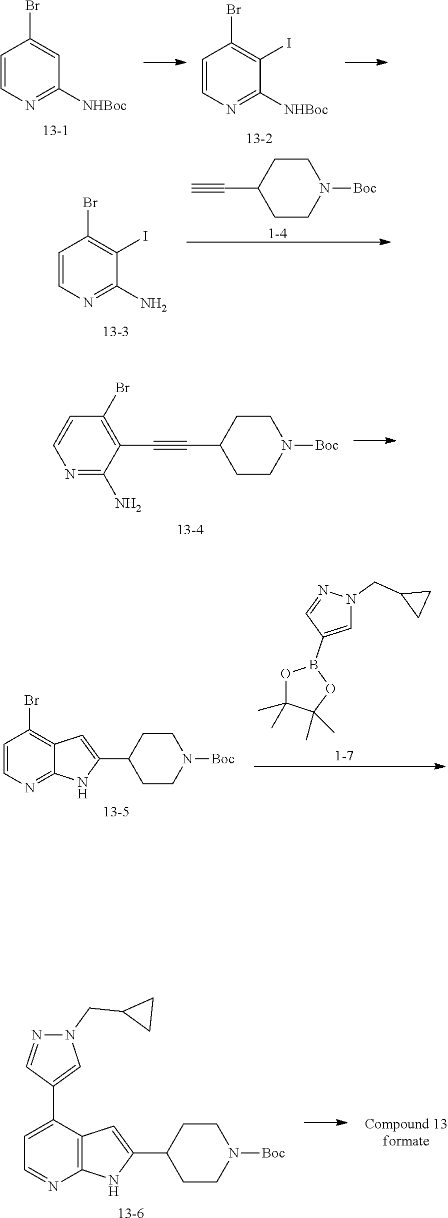

### Step 1:

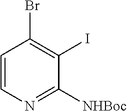 [SMILES: CC(C)(C)OC(=O)Nc1nccc(Br)c1I]

To solution of diisopropylamine (926.22 mg, 9.15 mmol, 1.29 mL, 2.5 equivalent) in tetrahydrofuran (5 mL) was added n-butyl lithium (2.5 mol/L, 3.66 mL, 2.5 equivalent) solution under the protection of nitrogen at −50° C., and the mixture was stirred at 0° C. for 0.5 hours. The mixture was added dropwise to 13-1 (1 g, 3.65 mmol, 1.0 equivalent) in tetrahydrofuran (5 mL) under nitrogen protection at −78° C., and the mixture was stirred at −78° C. for 1 hour. Then iodine (1.02 g, 4.03 mmol, 1.1 equivalent) dissolved in tetrahydrofuran (2 mL) was added dropwise to the reaction mixture, and the temperature was raised to 15° C. within half an hour. TLC (petroleum ether:ethyl acetate=5:1) showed that the starting material remained. The reaction mixture was poured into a saturated ammonium chloride (30 mL) solution, extracted with ethyl acetate (30*2 mL), the organic phase was washed with brine (30 mL), and then concentrated under reduced pressure. The resulting residue was purified by silica gel column (petroleum ether:ethyl acetate=5:1) to obtain compound 13-2.

### Step 2:

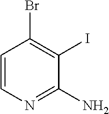

To solution of compound 13-2 (4(0) mg, 1.00 mmol, 1.0 equivalent) in ethyl acetate (10 mL) was added hydrogen chloride/ethyl acetate (4.0 mol/L, 10 mL, 39.90 equivalent), and the mixture was reacted at 15° C. for 0.5 hour. TLC (petroleum ether:ethyl acetate=5:1) panel showed that the starting material remained, and LCMS showed that the product was formed. The mixture was allowed to continue to react at 15° C. for 0.5 hours. TLC (petroleum ether:ethyl acetate=5:1) showed that the reaction of the starting material was complete. The reaction mixture was concentrated under reduced pressure to obtain crude product 13-3 for use directly in the next step. LCMS (ESI)/300.8 (M+1).

### Step 3:

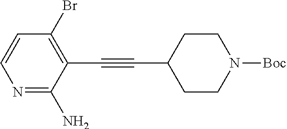 [SMILES: CC(C)(C)OC(=O)N1CCC(C#Cc2c(Br)ccnc2N)CC1]

To solution of 13-3 (0.37 g, 1.10 mmol, 1.0 equivalent) and 1-4 (230.90 mg, 1.10 mmol, 1.0 equivalent) in triethylamine (10 mL) was added bis(triphenylphosphine)palladium dichloride (154.88 mg, 220.65 μmol, 0.2 equivalent) and cuprous iodide (230.90 mg, 1.10 mmol, 1.0 equivalent) under the protection of nitrogen, and the mixture was stirred at 100° C. for 12 hours. LCMS showed that the reaction of the starting material was complete. The reaction solution was filtered with celite, the filter cake was washed with ethyl acetate (30 mL), the filtrate was concentrated under reduced pressure, and the resulting residue was purified by silica gel column (petroleum ether:ethyl acetate=1:1) to obtain compound 13-4. LCMS (ESI) m/z: 382.1 (M+1).

### Step 4:

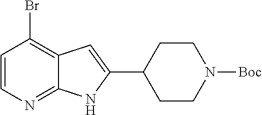 [SMILES: CC(C)(C)OC(=O)N1CCC(c2cc3c(Br)ccnc3[nH]2)CC1]

To solution of 13-4 (150 mg, 394.45 μmol, 1.0 equivalent) in N,N-dimethylformamide (5 mL) was added potassium tert-butoxide (132.79 mg, 1.18 mmol, 3 equivalent) at 15° C., and the mixture was stirred at 100° C. for 12 hours. TLC (petroleum ether:ethyl acetate=1:1) showed that the reaction of the starting material was complete. The reaction solution was poured into water (20 mL), extracted with ethyl acetate (20 mL*2), and the organic phase was concentrated under reduced pressure to obtain crude product 13-5 for use directly in the next step.

### Step 5:

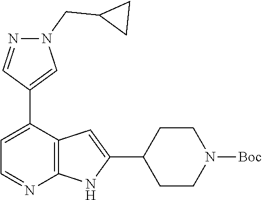 [SMILES: CC(C)(C)OC(=O)N1CCC(c2cc3c(-c4cnn(CC5CC5)c4)ccnc3[nH]2)CC1]

To mixture of 13-5 (130 mg, 341.85 μmol, 1.0 equivalent) and 1-7 (254.47 mg, 1.03 mmol, 3.0 equivalent) in dioxane (8 mL) and water (2 mL) was added Pd(dppf)Cl₂ (50.03 mg, 68.37 μmol, 0.2 equivalent) and cesium carbonate (334.05 mg, 1.03 mmol, 3 equivalent) under the protection of nitrogen at 25° C., and the mixture was stirred at 100° C. for 12 hours. TLC (petroleum ether:ethyl acetate=1:1) showed that the reaction of the starting material was complete. The reaction solution was filtered with celite, the filter cake was washed with ethyl acetate (30 mL), the filtrate was concentrated under reduced pressure, and the obtained residue was purified by silica gel column (petroleum ether:ethyl acetate:=1:1) to obtain compound 13-6. LCMS (ESI) m/z: 422.2 (M+1).

### Step 6:

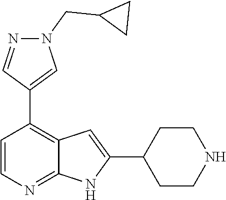 [SMILES: c1cc(-c2cnn(CC3CC3)c2)c2cc(C3CCNCC3)[nH]c2n1]

To solution of compound 13-6 (150 mg, 313.36 μmol, 1.0 equivalent) in ethyl acetate (5 mL) was added hydrogen chloride/ethyl acetate (4.0 mol/L, 5 mL, 63.83 equivalent), and the mixture was reacted at 15° C. for 15 minutes. LCMS showed that the reaction of the starting material was complete. The reaction mixture was concentrated under reduced pressure, the residue was dissolved in water (15 mL), the pH of the aqueous phase was adjusted to 9 with saturated sodium carbonate solution, extracted with ethyl acetate (20 mL*2), and the organic phase was concentrated under reduced pressure. The obtained residue was purified by preparative HPLC (chromatographic column: Shim-pack C18 150*25 mm*0 μm; formic acid, mobile phase: [water (0.225% formic acid)-acetonitrile], gradient: acetonitrile 0%-26%) to obtain the formate of compound 13. ¹H NMR (400 MHz, DMSO-d₆) δ 11.64 (br s, 1H), 8.49-8.45 (m, 1H), 8.47 (s, 1H), 8.35 (br s, 1H), 8.11-8.06 (m, 2H), 7.22 (d, J=5.0 Hz, 1H), 6.51 (s, 1H), 4.06 (d, J=7.2 Hz, 2H), 3.32 (br d, J=12.4 Hz, 2H), 3.08-2.90 (m, 1H), 3.08-2.90 (m, 3H), 2.21 (br d, J=12.4 Hz, 2H), 1.95-1.79 (m, 2H), 1.38-1.24 (m, 1H), 0.60-0.52 (m, 2H), 0.45-0.35 (m, 2H); LCMS (ESI) m/z: 322.1 (M+1).

### Example 14

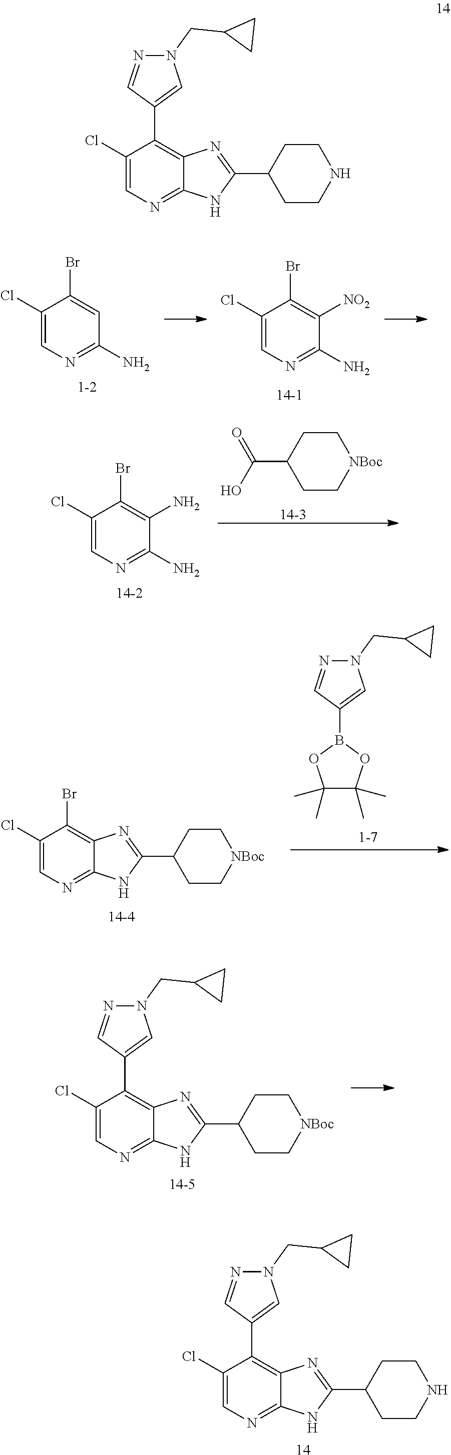

### Step 1:

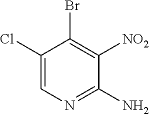 [SMILES: Nc1ncc(Cl)c(Br)c1[N+](=O)[O-]]

To solution of compound 1-2 (5.0 g, 24.10 mmol, 1.0 equivalent) in concentrated sulfuric acid (60 mL) was added potassium nitrate (3.66 g, 36.15 mmol, 1.5 equivalent) in portions at 0° C. The mixture was reacted at 15° C. for 12 hours. TLC (petroleum ether:ethyl acetate=2:1) showed that the reaction of the starting material was complete. The reaction solution was slowly poured into ice water (80 mL), the mixture was filtered, and the filter cake was dried under reduced pressure to obtain compound 14-1.

### Step 2:

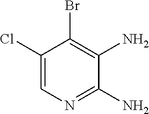

To solution of compound 14-1 (1.0 g, 3.96 mmol, 1.0 equivalent) in ethanol (40 mL) and water (20 mL) was added ammonium chloride (1.06 g, 19.81 mmol, 5.0 equivalent) and iron powder (66.63 mg, 11.88 mmol, 3.0 equivalent). The mixture was reacted at 80° C. for 2 hours, and LCMS showed that the reaction of the starting material was complete. The reaction solution was cooled to 15° C., filtered, and the filter cake was washed with ethanol (50 mL). The filtrate was concentrated under reduced pressure to obtain compound 14-2. LCMS (ESI) m/z: 224.0 (M+1).

### Step 3:

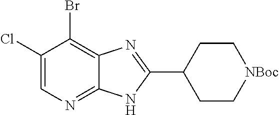 [SMILES: CC(C)(C)OC(=O)N1CCC(c2nc3c(Br)c(Cl)cnc3[nH]2)CC1]

To solution of compound 14-2 (4(0) mg, 1.80 mmol, 1.0 equivalent) in N,N-dimethylformamide (20 mL) was added compound 14-3 (618.34 mg, 2.70 mmol, 1.5 equivalent), carbonyl diimidazole (437.31 mg, 2.70 mmol, 1.5 equivalent) and pyridine (426.66 mg, 5.39 mmol, 435.37 μL, 3.0 equivalent). The mixture was reacted under the protection of nitrogen at 100° C. for 2 hours. LCMS showed that the starting material had not reacted completely. Potassium carbonate (585.82 mg, 1.80 mmol, 1.0 equivalent) was added to the reaction solution, and the mixture was reacted under the protection of nitrogen at 100° C. for 10 hours. LCMS showed that the reaction of the starting material was complete. The reaction solution was poured into water (50 mL), extracted with ethyl acetate (50 mL*2), the organic phases were combined, washed once with saturated brine (50 mL), the organic phase was separated, and concentrated under reduced pressure to obtain a residue. The residue was separated with a silica gel plate (petroleum ether:ethyl acetate=1:1) to obtain compound 14-4. LCMS (ESI) m/z: 417.0 (M+1).

### Step 4:

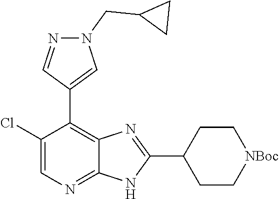 [SMILES: CC(C)(C)OC(=O)N1CCC(c2nc3c(-c4cnn(CC5CC5)c4)c(Cl)cnc3[nH]2)CC1]

To solution of compound 14-4 (180 mg, 383.37 μmol, 1.0 equivalent) in 1,4-dioxane (10 mL) and water (5 mL) was added compound 1-7 (190.25 mg, 766.74 μmol, 2.0 equivalent), Pd(dppf)Cl₂ (62.62 mg, 76.67 μmol, 0.2 equivalent) and cesium carbonate (374.73 mg, 1.15 mmol, 3.0 equivalent). After the mixture was replaced with nitrogen three times, it was allowed to react at 110° C. for 2 hours. LCMS showed that the reaction of the starting material was complete. The reaction solution was cooled to 15° C., and concentrated under reduced pressure to obtain a residue. The residue was extracted with ethyl acetate (50 mL*2), the organic phases were combined and washed with saturated brine (100 mL). The organic phase was separated, and concentrated under reduced pressure to obtain a residue. The residue was separated by a silica gel plate (petroleum ether:ethyl acetate=1:1) to obtain compound 14-5. LCMS (ESI) m/z: 457.2 (M+1).

### Step 5:

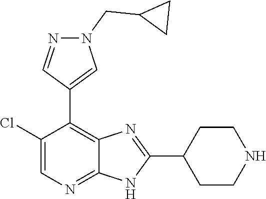 [SMILES: Clc1cnc2[nH]c(C3CCNCC3)nc2c1-c1cnn(CC2CC2)c1]

To solution of compound 14-5 (100 mg, 212.27 μmol, 1.0 equivalent) in ethyl acetate (5 mL) was added a hydrogen chloride/ethyl acetate solution (4 mol/L, 5.0 mL, 94.22 equivalent). The mixture was allowed to continue to react at 15° C. for 0.5 hours, and LCMS showed that the reaction of the starting material was complete. The reaction solution was concentrated to obtain a residue. The residue was dissolved in water (20 mL). The pH was adjusted to 8 with an aqueous solution of sodium bicarbonate (w %=10%). The mixture was extracted with ethyl acetate (20 mL*2), the organic phases were combined, and concentrated under reduced pressure to obtain a residue. The residue was purified by preparative HPLC (chromatographic column: Boston Green ODS 150*30 mm*5 μm; formic acid, mobile phase: water (0.225% formic acid)-acetonitrile, gradient: acetonitrile 10%-40%) to obtain compound 14. ¹H NMR (400 MHz, CD₃OD) δ 8.66 (s, 1H), 8.51 (s, 1H), 8.45 (s, 1H), 8.30 (s, 1H), 4.13 (d, J=7.2 Hz, 2H), 3.56 (td J=3.7, 12.9 Hz, 2H), 3.40-3.32 (m, 1H), 3.26-3.15 (m, 2H), 2.42-2.31 (m, 2H), 2.28-2.14 (m, 2H), 1.45-1.32 (m, 1H), 0.71-0.64 (m, 2H), 0.51-0.44 (m, 2H); LCMS (ESI) m/z: 357.2 (M+1).

### Example 15

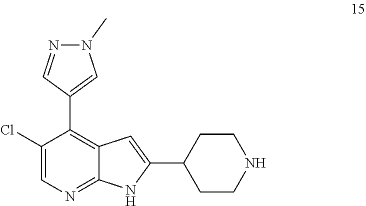 [SMILES: Cn1cc(-c2c(Cl)cnc3[nH]c(C4CCNCC4)cc23)cn1]

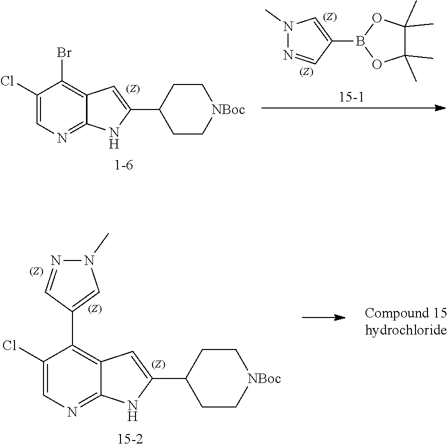

### Step 1:

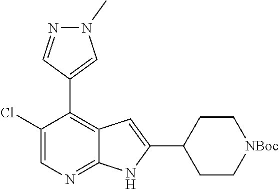 [SMILES: Cn1cc(-c2c(Cl)cnc3[nH]c(C4CCN(C(=O)OC(C)(C)C)CC4)cc23)cn1]

To solution of compound 1-6 (0.5 g, 1.21 mmol, 1 equivalent) and compound 15-1 (376.27 mg, 1.81 mmol, 1.5 equivalent) in dioxane (20 mL) and water (5 mL) was added Pd(dppf)Cl₂.CH₂Cl₂ (196.91 mg, 241.12 μmol, 0.2 equivalent) and cesium carbonate (1.18 g, 3.62 mmol, 3 equivalent). The mixture was reacted at 100° C. for 12 hours. TLC (petroleum ether:ethyl acetate=1:1) showed that the reaction of the starting material was complete, and the reaction solution was concentrated to obtain a residue. The residue was purified by column chromatography (silica, eluent: petroleum ether:ethyl acetate=1:1) to obtain compound 15-2. LCMS (ESI) m/z: 416.2 (M+1).

### Step 2:

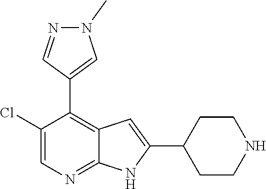 [SMILES: Cn1cc(-c2c(Cl)cnc3[nH]c(C4CCNCC4)cc23)cn1]

To solution of compound 15-2 (0.4 g, 961.73 mmol, 1 equivalent) in ethyl acetate (10 mL) was added hydrogen chloride/ethyl acetate (4 mol/L, 10 mL, 41.59 equivalent). The reaction solution was reacted at 15° C. for 15 minutes, and LCMS showed that the reaction of the starting material was complete. The reaction solution was concentrated to obtain a residue, and the residue was purified by preparative HPLC [chromatographic column: Phenomenex luna C18 150*40 mm*15 μm; water (0.05% hydrochloric acid)-acetonitrile: 7%-37%, 10 minutes] to obtain the hydrochloride of compound 15. ¹H NMR (400 MHz, DMSO-d₆) δ 11.96 (s, 1H₁), 9.21 (br d, J=8.8 Hz, 1H), 8.97 (br d, J=10.2 Hz, 1H), 8.31 (s, 1H), 8.19 (s, 1H), 7.96 (s, 1H), 6.34 (d, J=1.4 Hz, 1H), 3.96 (s, 3H), 3.33 (br d, J=12.4 Hz, 2H), 3.13-2.94 (m, 3H), 2.24 (br d, J=12.2 Hz, 2H), 1.98-1.81 (m, 2H); LCMS (ESI) m/z: 316.2 (M+1).

### In Vitro Activity Test

The compounds of the present invention are CDK9 inhibitors. The following experimental results confirm that these compounds listed in this patent are indeed CDK9 inhibitors and can be used as potential anticancer drugs. The IC₅₀ used in this patent refers to the concentration of the corresponding reagent when a certain reagent produces 50% maximum inhibition concentration.

### Experiment 1: In Vitro CDK9/CyclinT1 Enzyme Activity Test

### Experimental Materials:

CDK9/CyclinT1 kinase was purchased from Carna, ADP-Glo detection kit was purchased from Promega, and PKDTide substrate and kinase reaction buffer were purchased from Signalchem. Nivo multi-label analyzer (PerkinElmer).

### Experimental Method:

The kinase buffer in the kit was used to dilute the enzyme, substrate, adenosine triphosphate and inhibitors.

The compound to be tested was diluted by 5 times for 8 concentrations with a discharge gun, ie., dilution from 50 μM to 0.65 nM, with a DMSO concentration of 5%, and replicated. 1 μL of each concentration gradient of the inhibitor, 2 μL CDK9/CyclinT1 enzyme (4 ng), 2 μL of a mixture of substrate and ATP (100 μM adenosine triphosphate, 0.2 μg/μL substrate) was added to the microplate, and the final concentration gradient of the compound was 10 μM dilution to 0.13 nM. The reaction system was placed at 25° C. for 120 minutes. After the reaction, 5 μL of ADP-Glo reagent was added to each well, and the reaction was allowed to continue at 25° C. for 40 minutes. After the reaction, 10 μL of kinase detection reagent was added to each well. After reacting at 25° C. for 30 minutes, a multi-label analyzer was used to read chemiluminescence. The integration time was 0.5 seconds.

### Data Analysis:

The original data is converted to the inhibition rate using the equation (Sample−Min)/(Max−Min)*100%, and the value of IC₅₀ can be obtained by curve fitting with four parameters (log(inhibitor) vs. response−Variable slope mode in GraphPad Prism). Table 1 provides the enzymatic inhibitory activity of the compounds of the present invention on CDK9/CyclinT1.

### Experimental Conclusion:

The compounds of the present invention had good activity on CDK9 kinase. The activity was similar to the reference compounds BAY1251152 and AZD4573.

### Experiment 2: In Vitro CDK1/CyclinB1 Enzyme Activity Test

### Experimental Materials:

CDK1/CyclinB1 kinase detection kit was purchased from Promega. Nivo multi-label analyzer (PerkinElmer).

### Experimental Method:

Using the kinase buffer in the kit to dilute the enzyme, substrate, adenosine triphosphate and inhibitor.

The compound to be tested was diluted 5-fold to the 8th concentration with a discharge gun, that is, diluted from 50 μM to 0.65 nM, with a DMSO concentration of 5%, and replicated. Add 1 μL of each inhibitor concentration gradient, 2 μL CDK1/CyclinB1 enzyme (12.5 ng), 2 μL mixture of substrate and ATP (25 μM adenosine triphosphate, 0.2 μg/μL substrate) into the microplate, and the final concentration gradient of the compound is 10 μM dilute to 0.13 nM. The reaction system was placed at 25° C. for 120 minutes. After the reaction, add 5 μL of ADP-Glo reagent to each well, and continue the reaction at 25° C. for 40 minutes. After the reaction, add 10 μL of kinase detection reagent to each well. After reacting at 25° C. for 30 minutes, use a multi-label analyzer to read chemiluminescence. The integration time is 0.5 seconds.

### Data Analysis:

Using the equation (Sample−Min)/(Max−Min)*100% to convert the original data into the inhibition rate, the IC50 value can be obtained by curve fitting with four parameters (log(inhibitor) vs. response−Variable slope mode in GraphPad Prism). Table 1 provides the CDK1/CyclinB1 enzymatic inhibitory activity of the compounds of the present invention.

### Experimental Conclusion:

The compounds of the present invention did not have strong inhibitory activity on CDK1 kinase. Therefore, the compounds of the present invention showed a better selectivity for CDK1 than BAY1251152 and AZD4573.

### Experiment 3: In Vitro CDK2/CyclinE1 Enzyme Activity Test

### Experimental Materials:

CDK2/CyclinE1 kinase detection kit was purchased from Promega. Nivo multi-label analyzer (PerkinElmer).

### Experimental Method:

Using the kinase buffer in the kit to dilute the enzyme, substrate, adenosine triphosphate and inhibitor.

The compound to be tested was diluted 5-fold to the 8th concentration with a discharge gun, that is, diluted from 50 μM to 0.65 nM, with a DMSO concentration of 5%, and replicated. Add 1 μL of each inhibitor concentration gradient, 2 μL CDK2/CyclinE1 enzyme (2 ng), 2 μL mixture of substrate and ATP (150 μM adenosine triphosphate, 0.1 μg/μL substrate) to the microplate, and the final concentration gradient of the compound is 10 μM dilution to 0.13 nM. The reaction system was placed at 25° C. for 60 minutes. After the reaction, add 5 μL of ADP-Glo reagent to each well, and continue the reaction at 25° C. for 40 minutes. After the reaction, add 10 μL of kinase detection reagent to each well. After reacting at 25° C. for 30 minutes, use a multi-label analyzer to read chemiluminescence. The integration time is 0.5 seconds.

### Data Analysis:

Using the equation (Sample−Min)/(Max−Min)*100% to convert the original data into the inhibition rate, the IC50 value can be obtained by curve fitting with four parameters (log(inhibitor) vs. response−Variable slope mode in GraphPad Prism). Table 1 provides the CDK2/CyclinE1 enzymatic inhibitory activity of the compounds of the present invention.

### Experimental Conclusion:

The compounds of the present invention did not have strong inhibitory activity on CDK2 kinase. Therefore, the compounds of the present invention showed a better selectivity for CDK2 than BAY1251152 and AZD4573.

### Experiment 4: In Vitro Cell Activity Test

### Experimental Materials:

IMDM medium, fetal bovine serum, penicillin/streptomycin antibiotics were purchased from Promega (Madison, Wis.). The MV-4-11 cell line was purchased from the Cell Bank of the Chinese Academy of Sciences. Nivo multi-label analyzer (PerkinElmer).

### Experimental Method:

Plant MV-4-11 cells in a white 96-well plate, 80 μL of cell suspension per well, which contains 6000 MV-4-11 cells. The cell plate was cultured overnight in a carbon dioxide incubator.

The compound to be tested was diluted 5-fold to the 8th concentration with a discharge gun, that is, diluted from 2 mM to 26 nM, and replicated. Add 78 μL of culture medium to the middle plate, and then transfer 2 μL of each well of the gradient dilution compound to the middle plate according to the corresponding position, and transfer 20 μL of each well to the cell plate after mixing. The final concentration of the compound is 10 μM to 0.13 nM. The cell plate was placed in a carbon dioxide incubator for 3 days.

Add 25 μL of Promega CellTiter-Glo reagent per well to the cell plate and incubate at room temperature for 10 minutes to stabilize the luminescence signal. Use PerkinElmer Nivo multi-label analyzer to read.

### Data Analysis:

Using the equation (Sample−Min)/(Max−Min)*100% to convert the original data into the inhibition rate, the IC₅₀ value can be obtained by curve fitting with four parameters (log(inhibitor) vs. response−Variable slope mode in GraphPad Prism). Table 1 provides the inhibitory activity of the compounds of the present invention on the proliferation of MV-4-11 cells.

### Experimental Conclusion:

The compounds of the present invention had good cell anti-proliferation activity against MV4-11.

TABLE 1

| | CDK9 | CDK1 | CDK2 | MV4-11 Cell |
|---|---|---|---|---|
| Test Compound | IC₅₀ (nM) | IC₅₀ (nM) | IC₅₀ (nM) | IC₅₀ (nM) |
| BAY1251152 | 5.1 | 299.9 | 193.0 | 28.2 |
| AZD4573 | 3.0 | 2.0 | 1.0 | 14.5 |
| Compound 1 | 7.7 | 88.7 | 112.3 | 14.7 |
| (hydrochloride) | | | | |
| Compound 2 (formate) | 7.1 | 254.8 | 79.4 | 35.7 |
| Compound 3 | 14.1 | 415.7 | 104.9 | 7.4 |
| (hydrochloride) | | | | |
| Compound 4 | 10.3 | 756.1 | 333.9 | 94.8 |
| (hydrochloride) | | | | |
| Compound 5 | 10.6 | 1548 | 235.5 | 63.8 |
| (hydrochloride) | | | | |
| Compound 6 (formate) | 9.6 | 1029.0 | 393.6 | 52.0 |
| Compound 7 | 5.2 | 366.1 | 112.5 | 86.6 |
| Compound 8 (formate) | 7.1 | 177.7 | 75.0 | 54.6 |
| Compound 9 (formate) | 6.3 | 132.7 | 105.2 | 67.3 |
| Compound 10 (formate) | 6.9 | 87.9 | 549.3 | 79.1 |
| Compound 11 (formate) | 6.4 | 120.2 | 214.2 | 64.8 |
| Compound 12 | 7.7 | 260.2 | 549.4 | 286.0 |
| Compound 3 (formate) | 18.0 | 879.1 | 932.7 | 251.8 |
| Compound 14 | 38.4 | — | 1363.0 | 464.2 |
| Compound 15 | 4.3 | 823.7 | 684.9 | 34.5 |
| (hydrochloride) | | | | |

### Experiment 5: In Vivo Drug Efficacy Study (1)

In vivo drug efficacy experiments were performed on human-derived tumor cell line-based xenograft (CDX) BALB/c nude mice derived from patients with MV4-11 acute myeloid leukemia subcutaneously implanted.

### Experimental Operation:

Female 6- to 8-week old BALB/c nude mice which weighed about 18-22 g were kept in separate ventilated cages (3 mice per cage) in a special pathogen-free environment. All the cages, matts and water were disinfected before use. All the animals had free access to standard certified commercial laboratory diet. A total of 30 mice were purchased from Shanghai Lingchang biological science and technology Co., LTD. for study. Each mouse was subcutaneously implanted with tumor cells in the right next abdomen (10×10⁶ in 0.2 mL of phosphate buffer) for tumor growth. The administration started when the average tumor volume reached approximately 110 cubic millimeters. The test compound was injected weekly at a dose of 15 mg/kg. The tumor volumes were measured with a two-dimensional caliper twice a week, and the volumes were expressed in cubic millimeters and calculated by the formula: V=0.5a×b², where a and b were the major diameter and the minor diameter of the tumor, respectively. Antitumor efficacy was determined by dividing an average increase of the tumor volumes of the animals treated with the compounds by an average increase of the tumor volumes of the untreated animals.

### Experimental Conclusion:

In the CDX in vivo pharmacodynamic model of MV4-11 acute myeloid leukemia, the compound of the present invention exhibited good pharmacodynamics and safety.

TABLE 2

| | | Animals' | | | | |
|---|---|---|---|---|---|---|
| | Dosage | Drug | Tumor Volume (mm³) | | | |
| Compound | (mg/kg) | Tolerance | Day 0 | Day 7 | Day 17 | Day 28 |
| Blank | 0 | Well | 110 | 233 | 480 | 1277 |
| AZD4573 | 15 | Well | 110 | 173 | 262 | 975 |
| BAY1251152 | 15 | 5/6 mouse | 110 | 107 | 83 | 426 |
| | | death | | | | |
| Compound 1 | 15 | Well | 110 | 109 | 42 | 107 |
| (hydrochloride) | | | | | | |

### Experiment 6: In Vivo Drug Efficacy Study (2)

In vivo drug efficacy experiments were performed on human-derived tumor cell line-based xenograft (CDX) BALB/c nude mice derived from patients with MV4-1 acute myeloid leukemia subcutaneously implanted.

### Experimental Operation:

Female 6- to 8-week old BALB/c nude mice which weighed about 18-22 g were kept in separate ventilated cages (3 mice per cage) in a special pathogen-free environment. All the cages, matts and water were disinfected before use. All the animals had free access to standard certified commercial laboratory diet. A total of 36 mice were purchased from Shanghai Lingchang biological science and technology Co., LTD. for study. Each mouse was subcutaneously implanted with tumor cells in the right next abdomen (10×10⁶ in 0.2 mL of phosphate buffer) for tumor growth. The administration started when the average tumor volume reached approximately 121 cubic millimeters. The test compound was injected weekly at a dose of 10 mg/kg. The tumor volumes were measured with a two-dimensional caliper twice a week, and the volumes were expressed in cubic millimeters and calculated by the formula: V=0.5a×b², where a and b were the major diameter and the minor diameter of the tumor, respectively. Antitumor efficacy was determined by dividing an average increase of the tumor volumes of the animals treated with the compounds by an average increase of the tumor volumes of the untreated animals.

### Experimental Conclusion:

In the CDX in vivo pharmacodynamic model of MV4-11 acute myeloid leukemia, the compounds of the present invention exhibited good pharmacodynamics and safety.

TABLE 3

| | | Animals' | | | | |
|---|---|---|---|---|---|---|
| | Dosage | Drug | Tumor Volume (mm³) | | | |
| Compund | (mg/kg) | Tolerance | Day 0 | Day 7 | Day 17 | Day 28 |
| Blank | 0 | Well | 121 | 198 | 678 | 1643 |
| Compound 1 | 10 | Well | 121 | 121 | 151 | 419 |
| (hydrochloride) | | | | | | |
| Compound 15 | 10 | Well | 121 | 87 | 74 | 332 |
| (hydrochloride) | | | | | | |

What is claimed is:

1. A compound represented by the formula (I), or a pharmaceutically acceptable salt or an isomer thereof, 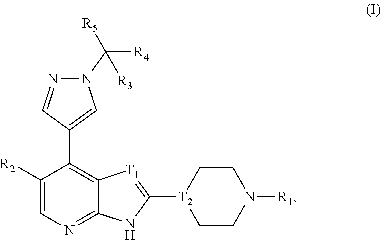
wherein, T₁ is N or CR;
R is H or Cl;
T₂ is N or CH;
R₁ is H or C₁₋₆ alkyl, wherein the C₁₋₆ alkyl is optionally substituted with 1, 2 or 3 substituents independently selected from F, Cl, —OH, —NH₂ and C₁₋₃ alkoxy;
R₂ is H, F or Cl;
R₃ and R₄ are each independently H, F, Cl or C₁₋₃ alkyl;
R₅ is H, C₃₋₆ cycloalkyl or phenyl, wherein the C₃₋₆ cycloalkyl and phenyl are optionally substituted with 1, 2 or 3 Rₐ;
each Rₐ is independently H, F, Cl, C₁₋₃ alkyl or C₁₋₃ alkoxy.

2. The compound of claim 1, or a pharmaceutically acceptable salt or an isomer thereof, and said compound has a structure represented by formula (I-1) or (I-2): 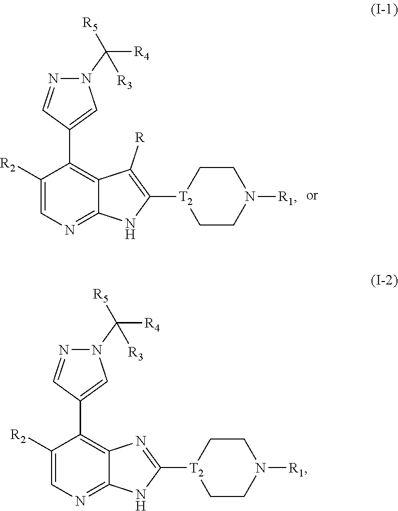
wherein, R, T₂, R₁, R₂, R₃, R₄ and R₅ are as defined in claim 1.

3. The compound of claim 2, or a pharmaceutically acceptable salt or an isomer thereof, and said compound has a structure represented by formula (I-1-a) or (I-1-b): 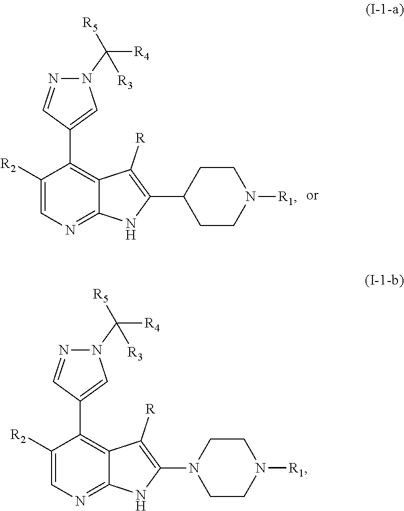
wherein, R, R₁, R₂, R₃, R₄ and R₅ are as defined in claim 2.

4. The compound of any one of claims 1-3, or a pharmaceutically acceptable salt or an isomer thereof, wherein, R₃ and R₄ are each independently H, F or

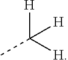

5. The compound of claim 2, or a pharmaceutically acceptable salt or an isomer thereof, and said compound has a structure represented by formula (I-1-c) or (I-1-d): 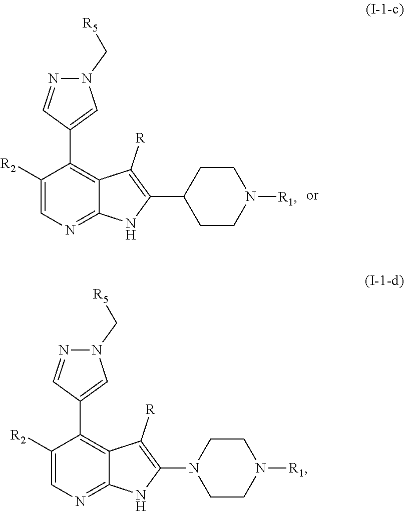
wherein, R, R₁, R₂ and R₅ are as defined in claim 2.

6. The compound of claim 1, or a pharmaceutically acceptable salt or an isomer thereof, wherein each Rₐ is independently H, F, Cl or

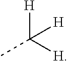

7. The compound of any one of claims 1-3, 5 or 6, or a pharmaceutically acceptable salt or an isomer thereof, wherein R₅ is H, 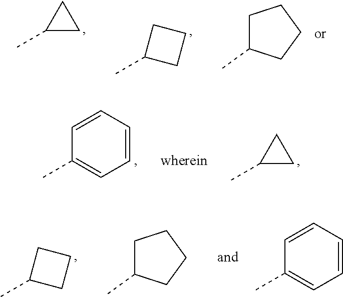
are optionally substituted with 1, 2 or 3 Rₐ.

8. The compound of claim 7, or a pharmaceutically acceptable salt or an isomer thereof, wherein R₅ is H,

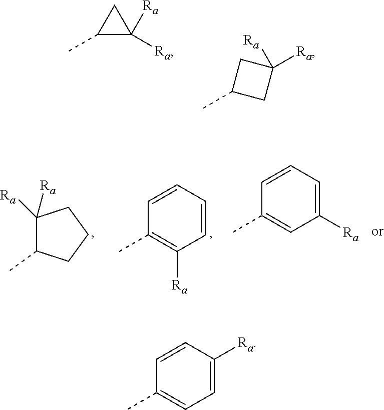

9. The compound of claim 8, or a pharmaceutically acceptable salt or an isomer thereof, wherein R₅ is H,

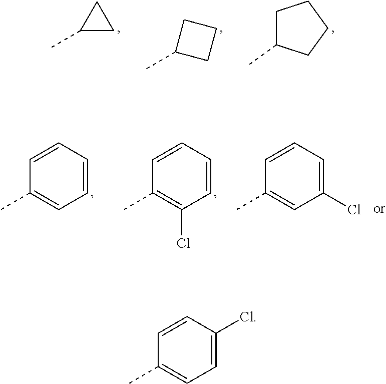

10. The compound of claim 7, or a pharmaceutically acceptable salt or an isomer thereof, and said compound has a structure represented by formula (I-1-e), (I-1-f), (I-1-g) or (I-1-h): 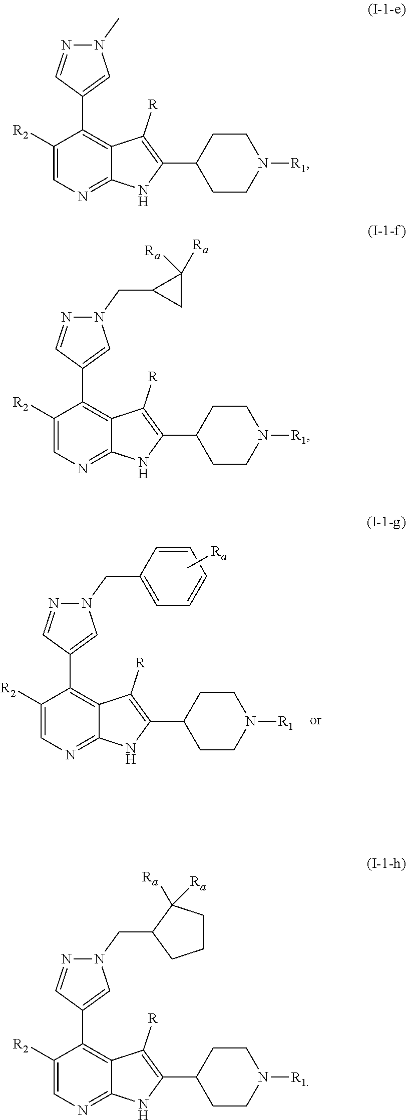
wherein, R, R₁, R₂ and Rₐ are as defined in claim 7.

11. The compound of any one of claims 1-3, 5 or 8-10, or a pharmaceutically acceptable salt or an isomer thereof, wherein R₁ is H, 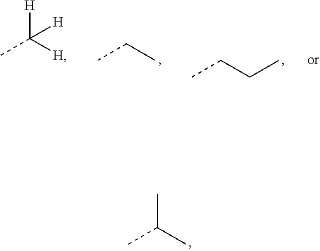
wherein the 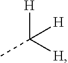 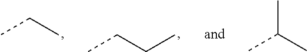
and are optionally substituted with 1, 2 or 3 substituents independently selected from F, Cl, —OH, —NH₂ and —OCH₃.

12. The compound of claim 11, or a pharmaceutically acceptable salt or an isomer thereof, wherein R₁ is H,

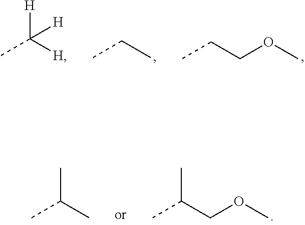

13. The compound of any one of claims 1-3, or a pharmaceutically acceptable salt or an isomer thereof, wherein the structural unit

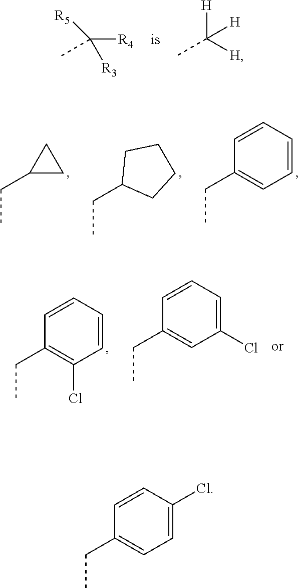

14. A compound, or a pharmaceutically acceptable salt or an isomer thereof, as follows:

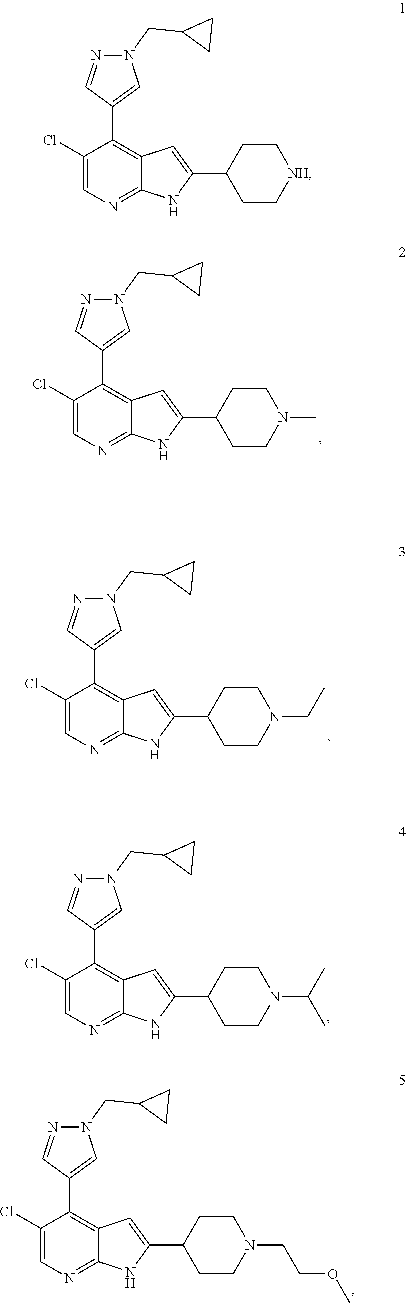 [SMILES: Clc1cnc2[nH]c(C3CCNCC3)cc2c1-c1cnn(CC2CC2)c1 ; CN1CCC(c2cc3c(-c4cnn(CC5CC5)c4)c(Cl)cnc3[nH]2)CC1 ; CCN1CCC(c2cc3c(-c4cnn(CC5CC5)c4)c(Cl)cnc3[nH]2)CC1 ; CC(C)N1CCC(c2cc3c(-c4cnn(CC5CC5)c4)c(Cl)cnc3[nH]2)CC1 ; COCCN1CCC(c2cc3c(-c4cnn(CC5CC5)c4)c(Cl)cnc3[nH]2)CC1]

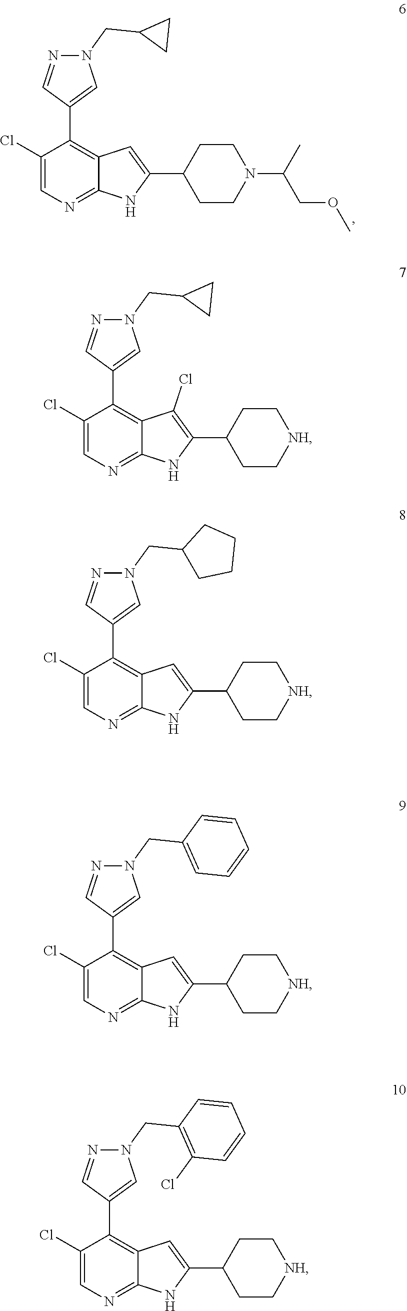 [SMILES: COCC(C)N1CCC(c2cc3c(-c4cnn(CC5CC5)c4)c(Cl)cnc3[nH]2)CC1 ; Clc1cnc2[nH]c(C3CCNCC3)c(Cl)c2c1-c1cnn(CC2CC2)c1 ; Clc1cnc2[nH]c(C3CCNCC3)cc2c1-c1cnn(CC2CCCC2)c1 ; Clc1cnc2[nH]c(C3CCNCC3)cc2c1-c1cnn(Cc2ccccc2)c1 ; Clc1ccccc1Cn1cc(-c2c(Cl)cnc3[nH]c(C4CCNCC4)cc23)cn1]

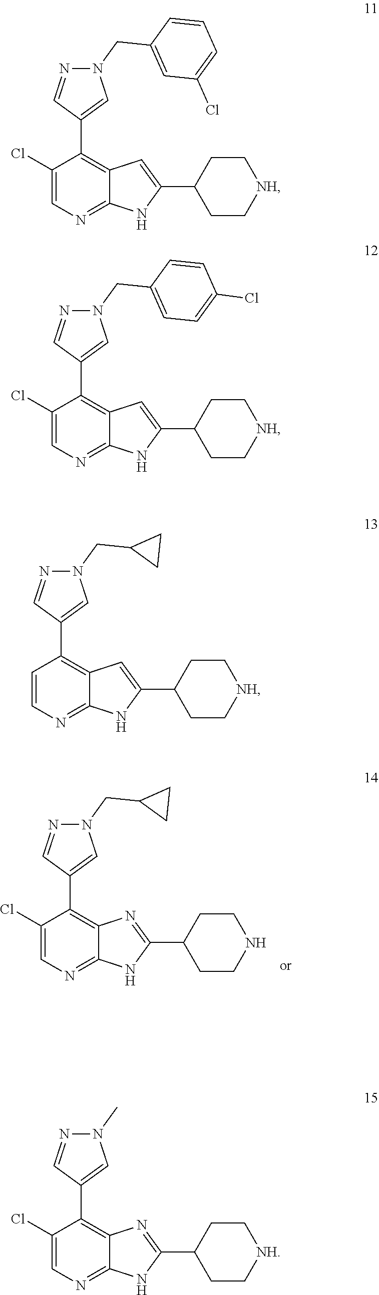 [SMILES: Clc1cccc(Cn2cc(-c3c(Cl)cnc4[nH]c(C5CCNCC5)cc34)cn2)c1 ; Clc1ccc(Cn2cc(-c3c(Cl)cnc4[nH]c(C5CCNCC5)cc34)cn2)cc1 ; c1cc(-c2cnn(CC3CC3)c2)c2cc(C3CCNCC3)[nH]c2n1 ; Clc1cnc2[nH]c(C3CCNCC3)nc2c1-c1cnn(CC2CC2)c1 ; Cn1cc(-c2c(Cl)cnc3[nH]c(C4CCNCC4)nc23)cn1]

15. The use of the compound of any one of claims 1-14, or a pharmaceutically acceptable salt or an isomer thereof in the preparation of a CDK9 inhibitor drug.

16. The use of the compound of any one of claims 1-14, or a pharmaceutically acceptable salt or an isomer thereof in the preparation of a medicine for the treatment of cancer.
